(19) United States
(12) Patent Application Publication
(10) Pub. No.: US 2026/0018245 A1
(43) Pub. Date: Jan. 15, 2026

(54) SYSTEMS AND METHODS FOR IDENTIFYING AND TARGETING COLLATERAL LETHAL GENES

(71) Applicant: The Regents of the University of Michigan, Ann Arbor, MI (US)

(72) Inventors: Olamide Animasahun, Ann Arbor, MI (US); Fulei Wuchu, Ann Arbor, MI (US); Minal Nenwani, Ann Arbor, MI (US); Abhinav Achreja, Ann Arbor, MI (US); Deepak Nagrath, Ann Arbor, MI (US)

(21) Appl. No.: 19/112,888

(22) PCT Filed: Sep. 21, 2023
(86) PCT No.: PCT/US2023/074767

§ 371 Date: Mar. 18, 2025

Related U.S. Application Data
(60) Provisional application No. 63/376,518, filed on Sep. 21, 2022.

(51) Int. Cl. G16B 20/20; G16B 45/00; G16H 20/10; G16H 50/20

(52) U.S. Cl. CPC G16B 20/20; G16B 45/00; G16H 20/10; G16H 50/20

(57) ABSTRACT

Provided herein are systems and methods for identifying collateral lethal genes via metabolic fluxes and the use of such genes for the identification of, selections of, and use of therapeutic agents. Further provided herein are identified targets, such as MTHFD2, that may be regulated to treat or prevent diseases and conditions.

The present application claims priority to U.S. Provisional Patent Application 63/376,518, filed Sep. 21, 2022, the disclosure of which is herein incorporated by reference in its entirety.

### STATEMENT REGARDING FEDERAL FUNDING

This invention was made with government support under CA204969, CA227622 and CA222251 awarded by the National Institutes of Health. The government has certain rights in the invention.

### FIELD

Provided herein are systems and methods for identifying collateral lethal genes via metabolic fluxes and the use of such genes for the identification of, selections of, and use of therapeutic agents. Further provided herein are identified targets, such as MTHFD2, that may be regulated to treat or prevent diseases and conditions.

### BACKGROUND

Recent work has leveraged the connection between genetic and epigenetic interactions regulating the development of tumorigenic phenotypes to identify cancer-specific vulnerabilities. Recurrent loss-of-function deletions are prevalent genomic alterations, cause frequent inactivation of tumour suppressor genes and are important driver events in tumorigenesisi. Although these deletions offer functional and fitness advantages to cancer cells, they make them vulnerable due to the collateral deletion of essential genes in chromosomal proximity²⁻⁷. Essential passenger gene deletions in cancer cells can concomitantly lead to dependence on paralogues that maintain a similar function. While the loss of genes that encode metabolic enzymes may block a metabolic pathway, redundancy in the metabolic circuitry and plasticity of cancer cells allows them to utilize alternative pathways not predictable from first principles. The emergence of these context-specific compensatory pathways gives rise to the concept of collateral lethality.

Although frameworks have been developed recently to uncover gene associations for coessentiality mapping and gene essentialitys⁸⁻¹², no current methodology can systematically uncover collateral lethal genes for deleted passenger metabolic genes. Identification of paralogues is crucial to exploiting the vulnerabilities of cancer cells while sparing normal cells.

Previous approaches identified these paralogous metabolic genes by considering the functional compensation occurring through enzyme isoforms^{2-4,13} While current approaches, including genetic loss-of-function screens—can reveal paralogues based on gene essentiality, there is a pressing need for improved methods.

### SUMMARY

In some embodiments, provided herein are systems and methods for identification of collateral lethal genes via metabolic fluxes, CLTM. These systems and methods provide a methodology that can (1) dissect the differential metabolism within subcellular compartments, (2) incorporate redox and biosynthetic balances maintained through inter-organelle shuttling and (3) use metabolic fluxes to map genetic information with phenotypic biological functions. This comprehensive methodology not only provides for robust identification of paralogues but also reveals interconnectedness of metabolic networks by considering multiple cellular objectives beyond just proliferation.

The system was used to reveal MTHFD2 as a collateral lethal gene in UQCR11-deleted ovarian tumours. We show that MTHFD2 has a non-canonical oxidative function to provide mitochondrial NAD+, and demonstrate the regulation of systemic metabolic activity by the paralogue metabolic pathway maintaining metabolic flux compensation. This UQCR11-MTHFD2 collateral lethality is confirmed in vivo, with MTHFD2 inhibition leading to complete remission of UQCR11-deleted ovarian tumours. Using CLIM's machine learning and genome-scale metabolic flux analysis, we elucidate the broad efficacy of targeting MTHFD2 despite distinct cancer genetic profiles co-occurring with UQCR11 deletion and irrespective of stromal compositions of tumours.

The electron transport chain (ETC) is a series of redox reactions (oxidative phosphorylation or OXPHOS) that occur in the inter membrane space of the mitochondria. It is responsible for the transfer of electrons across the donors and acceptors to maintain a proton gradient across the mitochondrial membranes. This gradient then drives the synthesis of ATP, a major energy currency in cells, and maintains a balance of redox factors NAD+ and NADH within the mitochondria. As OXPHOS is an essential source of ATP for rapidly proliferating cancer cells, mitochondrial dysfunction drives metabolic rewiring to meet bioenergetic demands. Such mitochondrial dysfunction also presents in other metabolic diseases leading to pathological phenotypes. When studying genetic profiles of ovarian cancer patients, we discovered ETC-deficiency in a subset of these patients. We have seen that in cells with impairment in ETC shift their reliance onto other metabolic pathways to maintain their energy requirements and NAD+, revealing a metabolic target specific to ETC-deficient cells. By integrating computation metabolic flux analysis, machine learning, in vitro metabolic tracing we demonstrate that MTHFD2 has a noncanonical oxidative function to provide mitochondrial NAD+. Normally, MTHFD2 is responsible for maintaining 5,10-methylene-THF and 10-formyl-THF production in the mitochondria to recycle THF and provide formate for nucleotide synthesis in the cytosol. This canonical function of MTHFD2 relies on reduction of NAD+ to NADH, however, with computational modeling and parallel stable-isotope tracing with deuterated glucose and serine we discovered that MTHFD2 acts noncanonically to oxidize NADH to NAD+, in order to compensate for the reduced NAD+ recycling via the ETC. Further, we provide functional validation by showing selective death of ovarian cancer cells with ETC-deficiency. Using mouse models of ovarian cancer, we show the complete remission of ovarian tumors with ETC-deficiency. This UQCR11-MTHFD2 collateral lethality is confirmed in vivo, with MTHFD2 inhibition leading to complete remission of UQCR11-deleted ovarian tumors. MTHFD2 has long been considered a potential metabolic target in many cancers due to its overexpression in cancer cells compared to healthy adult cells, coupling to folate metabolism-mediated functions, redox balance, and nucleotide metabolism. Despite a broad empirical knowledgebase, the successful use of MTHFD2 inhibitors in the clinic has seen limited progress in cancers. Our discovery has revealed a selectively targetable noncaonical function of MTHFD2 in the context mitochondrial dysfunction. As such, MTHFD2 provides a novel therapeutic target in not only cancers, but other diseases caused by mitochondrial dysfunction or natural aging that leads to mitochondrial impairment.

In some embodiments, provided herein are methods for identifying collateral lethal genes or gene products. In some embodiments, the methods comprising analyzing data derived from a cell having a first gene or gene product. In some embodiments, the cell has a mutation or other defect that causes reduced or eliminated expression of the first gene compared to equivalent cells lacking the mutation or other defect. In some embodiments, the analysis is carried out using a computer program whereby the computer program analyzes the data and generates processed data revealing the identity of one or more collateral lethal genes or gene products. In some embodiments, the processed data is displayed. In some embodiments, one or more collateral lethal genes or gene products is identified. In some embodiments, the one or more collateral lethal genes or gene products is from a paralogous metabolic pathway that provides metabolic flux compensation to the cell when the lethal gene has reduced or eliminated expression. In some embodiments, the reduced or eliminated expression of the first gene results in electron transport chain (ETC) impairment in the cell.

In some embodiments, a system comprising a processor comprising or running software that conducts one or more the methods described herein is provided.

Some portions of this description describe the embodiments of the technology in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Certain steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the technology may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

In some embodiments, provided herein are methods for treating a subject comprising: administering an MTHFD2 inhibitor to the subject. In some embodiments, the subject has an ETC impairment (e.g., a Complex 1 dysfunction). In some embodiments, the ETC impairment is caused by reduced UQCR11 expression in one or more cells or tissues relative to normal levels due to a defect (e.g., mutation) in one or more genes that impacts UQCR11 expression (e.g., copy loss of, or a mutation in, a UQCR11 gene) (i.e., lower UQCR11 expression levels compared to an equivalent cell not having the defect). In some embodiments, the method comprises identifying a subject having ECT impairment (e.g., Complex 1 dysfunction, e.g., reduced UQCR11 expression) and administering an MTHFD2 inhibitor to the subject (test and treat). In some embodiments, the MTHFD2 expression is measured at one or more time points after administration of an MTHFD2 inhibitor and treatment of the subject is modified accordingly. In some embodiments, the ECT impairment (e.g., Complex 1 dysfunction, e.g., reduced UQCR11 expression) is in an ovarian cell or tissue of the subject. In some embodiments, the ovarian cell is an ovarian cancer cell (e.g., a cell derived from an ovarian tumor tissue).

In some embodiments, the MTHFD2 inhibitor is a small molecule, an antisense or RNAi molecule, or a gene therapy molecule (e.g., CRISPR system that modifies or deletes MTHFD2). In some embodiments, the MTHFD2 inhibitor is one or more or a combination of LY345899, DS44960156, DS18561882, (4-(3-(2,4-Diamino-6-oxo-1,6-dihydropyrimidin-5-yl)ureido)benzoyl)-L-glutamic acid, and inhibitors described in US20210047332, US20180370972, or WO2017023894 herein incorporated by reference in their entireties.

In some embodiments, MTHFD2 inhibitors are used in combination with other agents. For example, in some embodiments, MTHFD2 inhibitors are used in combination with other cancer therapeutic agents or modalities (e.g., surgery, radiation therapy, chemotherapy, hormone therapy, immunotherapy).

Further provided herein are methods comprising measuring MTHFD2 expression in a cell having ETC impairment. In some embodiments, the ETC impairment is caused by reduced or eliminated expression of a gene that is not MTHFD2 (e.g., UQCR11). In some embodiments, the cell is an ovarian cell. Such measuring finds use in research, drug screening, and drug selection applications. For treatment selection, in some embodiments, a high level of MTHFD2 expression and a ETC impairment (e.g., caused by a low level of UQCR11 expression) identifies cells, tissues, or subjects that are susceptible to treatment with an MTHFD2 inhibitor.

### DESCRIPTION OF FIGURES

FIG. 1 CLIM: a systems, multiomics and machine learning integrated-precision oncology platform that identifies collateral lethal metabolic targets. a,b, Circos plots showing average genomic copy number gain and loss, most significant focal deletions in chromosomes 1-22 (identified by GISTIC2.0) and number of total genes and metabolic gene symbols within deleted regions for (a) the TCGA Pan-Cancer patient dataset (n=10,104) and (b) the TCGA HGSOC dataset (n=606). c, Schematic describing the workflow used to (1) identify focal deletions in patients with particular genetic profiles, (2) determine the occurrence of metabolic vulnerabilities due to collateral loss of metabolic genes, (3) perform multi-objective, genome-scale metabolic flux analysis to reveal compensatory metabolic pathways that are collateral lethal targets and (4) test the validity of collateral lethal metabolic genes. d. Heatmap of scores obtained via machine learning NNMF algorithm to stratify ovarian tumours based on two molecular features: mutation status and CNA (specifically large-scale deletions). Each patient cluster (columns, vertical bar chart) is defined by a combination of molecular features wherein NNMF-derived cluster scores quantify the importance of molecular features (rows) in defining a patient cluster. Molecular features (rows) are sorted in descending order of the average of their scores (horizontal bar chart) across all patient clusters, representing the most important to the least important molecular features in ovarian tumours. CNA along chromosome 19 highlights copy number loss in 19p13.3 locus for five patient strata (clusters 3, 11, 12, 23 and 32), all of which have a high cluster score for the 19p13.3 deletion. CNA along chromosome 19 for cluster 21, which is defined by other molecular features and has the lowest cluster score for 19p13.3 deletion, shows no copy loss in the locus (CNA is approximately 0).

FIG. 2 Multi-objective metabolic flux analysis identifies MTHFD2 as a collateral lethal target in UQCR11-null ovarian cancers with in vitro, clinical and pan-cancer implications. a, Workflow elucidating multi-objective flux analysis algorithm comparing flux distributions of cancers with and without deletions for identification of collateral lethal pathways. b, Multi-objective metabolic Pareto frontier and metabolic fluxes of MTHFD1, MTHFD2, RNA and DNA syntheses in UQCR11-null and -intact models. Radar plots represent Pareto-optimal flux distributions from maximal biomass to maximal ATP maintenance (counterclockwise). c, mRNA expression of UQCR11 and MTHFD2 in parental ovarian cancer cell lines. Data presented as mean±s.d. (n=6), analysed using two-tailed Student's t-test. d, Protein expression of MTHFD2 and UQCR11. Representative immunoblots with β-actin as loading control. e, Cell viability after dox-induced shMTHFD2 knockdown. Data representative of two independent experiments and presented as mean±s.d. (n=3), analysed using two-way analysis of variance (ANOVA) with Dunn's correction. f, Viability of parental ovarian cancer lines treated with MTHFD2 inhibitor. Data representative of two independent experiments and presented as mean±s.d. (n=4). Data were fit to a three-parameter, variable-slope curve. g, Viability of isogenic SKOV3 lines expressing shScramble or shUQCR11, treated with MTHFD2 inhibitor. Data combined from two independent experiments and presented as mean±s.d. (n=6). Data were fit to a four-parameter, variable-slope curve. h, One-carbon and mitochondrial metabolism genes that predict progression-free survival in TCGA patients with ovarian cancer, based on 19p13.3 deletion status using random RSF. i, Correlation of mRNA expression of MTHFD2 and UQCR11 in 20 cancer types from TCGA Pan-Cancer dataset. Error bars represent 95% confidence intervals of correlation coefficients estimated with Spearman's rank correlation test. UQCR11 and MTHFD2 mRNA expression in TCGA UCEC, TCGA UCS tumours and CCLE endometrial cancer lines (n=612, top inset). MTHFD2 and UQCR11 mRNA expression and dependency scores of MTHFD2 (n=18, bottom inset). j, Overall survival of patients with UCEC and UQCR11 copy loss (below median) with high or low MTHFD2 mRNA expression (top and bottom quartiles), compared with patients with no copy loss (above median). Data analysed pairwise by log-rank test. k, Overall survival of patients with UCEC and low UQCR11 (bottom quartile) expression and high (top quartile) expression of MTHFD2 compared with patients with low UQCR11 and MTHFD2 (bottom quartile) expression. Data analysed by log-rank test. HR, hazard ratio.

FIG. 3 Metabolic flux analysis with isotope tracing using deuterium-labelled substrates demonstrates metabolic flux compensation through a paralogue pathway. a, In UQCR11-intact cells, mitochondrial MTHFD2 maintains a flux in the reductive direction converting NAD(P)+ to NAD(P)H while ETC, ALDH4A1 and AMT maintain NAD+/NADH pools oxidatively. b, Pathways in mitochondria, apart from ETC, that rely on NAD+/NADH include AMT, ALDH4A1 and MTHFD2. Directions of flux correspond to schematics (a,d), and the associated sign of flux values in Pareto-optimal flux space (c,e) are displayed. c, Pareto-optimal fluxes of reactions oxidizing NADH to NAD+, MTHFD2, AMT and ALDH4A1, as predicted by MOMFA in UQCR11-Intact cells, are plotted across the Pareto-optimal metabolic objectives of maximal growth and maximal survival. Fluxes are normalized to net pyruvate dehydrogenase (PDH) flux set to 100. d, In UQCR11-null cells, NAD+ depletion due to impaired ETC leads to MTHFD2 compensating for NAD+ depletion by maintaining a net flux in the oxidative direction, converting NAD(P)H to NAD(P)+. e, Pareto-optimal fluxes of reactions oxidizing NADH to NAD+, MTHFD2, AMT and ALDH4A1 as predicted by MOMFA in UQCR11-null cells are plotted across the Pareto-optimal metabolic objectives of maximal growth and maximal survival. Fluxes are normalized to net PDH flux set to 100. f, Stable-isotope tracing map elucidating the transfer of [2H] and enrichment of downstream metabolites from stable-isotope substrates 3-[2H]-glucose and 4-[2H]-glucose. g, Ratio of [2H]-serine/[2H]-NADH in UQCR11-null cells (OVCAR8, CAOV3 and HEYA8) relative to the same ratio in UQCR11-intact cells (SKOV3) after 24 h in 4-[2H]-glucose-labelled medium. Data combined from two independent experiments (n=6), with mean±s.d. estimated using error propagation and analysed by ordinary one-way ANOVA with Dunnett's correction for multiple comparisons. h, Isotopic enrichment of M+1 [2H]-serine in UQCR11-null cells relative to M+1 [2H]-serine enrichment in UQCR11-intact cells after 24 h in 3-[2H]-glucose-labelled medium. Data representative of two independent experiments, presented as mean±s.e.m. (n=3) and analysed by ordinary one-way ANOVA with Dunnett's correction for multiple comparisons.

FIG. 4 Dynamic flux analysis with parallel tracers to quantify oxidative MTHFD2 flux. a, Oxidative MTHFD2 flux and percentage decrease in OCRfollowing MTHFD2 inhibition in SKOV3 cells treated with rotenone (0, 31, 62, 125, 250, 500 or 1,000 nM) and MTHFD2 inhibitor (0 or 60 μM). Oxidative MTHFD2 flux is proportional to [2H1]-serine/[2H1]-NADH enrichment in cells cultured with 4-[2H]-glucose for 8 h. Percentage decrease in OCR is estimated in SKOV3 cells treated with 60 μM MTHFD2i compared with 0 μM MTHFD2i. Oxidative flux data are combined from two independent experiments (n=4), with mean±s.d. estimated using error propagation. OCR data are representative of two independent experiments and presented as mean±s.d. estimated using error propagation (n=5). b, Reductive MTHFD2 flux and basal OCR in SKOV3 cells treated with rotenone (0, 31, 62, 125, 250 or 1,000 nM) and MTHFD2 inhibitor (0 or 60 μM). Reductive MTHFD2 flux is proportional to [2H1]-NADH in cells cultured with 2,3,3-[2H]-serine for 8 h. OCR data are representative of two independent experiments and presented as mean±s.e.m. (n=5). c, Schematic of combination tracer experiment with 4-[2H]-glucose and [2H]-formate to track oxidative MTHFD2 flux. Inset: detailed view of [2H]transfer from 4-[2H]-glucose and [2H]-formate to MTHFD2 reaction intermediates, uniquely forming [2H2]-serine via oxidative MTHFD2. d, Fractional labelling of [2H2]-serine in SKOV3 and OVCAR8 cells at 0.5, 1 and 2 h following parallel tracer introduction of 4-[2H]-glucose and [2H]-formate. Data combined from two independent experiments, presented as mean±s.e.m. (n=6) and analysed using two-tailed Welch's t-test. e, Ratio of [2H2]-serine/[2H]-NADH enrichment in SKOV3 and OVCAR8 cells at 2 h after combined tracer introduction of 4-[2H]-glucose and [2H]-formate, representing the normalized contribution of [2H]-NADH to [2H2]-serine via oxidative MTHFD2 flux. Data combined from two independent experiments (n=6), with mean±s.d. estimated using error propagation and analysed by two-tailed Welch's t-test. f, [2H2]-serine enrichment in SKOV3 cells expressing shScramble or shUQCR11 at 2 h after combined tracer introduction of 4-[2H]-glucose and [2H]-formate. Enrichments relative to mean enrichment of [2H2]-serine in SKOV3 shScramble. Data combined from two independent experiments, presented as mean±s.e.m. (n=5) and analysed using one-tailed non-parametric Mann-Whitney test.

FIG. 5 Metabolic profiling and pathway analysis reveal metabolic compensation and systemic metabolic changes in UQCR11-intact and -null ovarian cancers, respectively, following inhibition of MTHFD2. a, Raindrop plot representing fold-change (FC) of individual metabolites present in each of the metabolic pathways as defined by the KEGG knowledge base. Circle colours represent log(FC) of metabolites in cells expressing shMTHFD2 with respect to shScramble in cell lines OVCAR8 (red) and SKOV3 (blue). Absolute log(FC) values are curtailed at 2.0 to improve visualization. Statistical analysis performed using two-sample t-tests corrected for multiple hypothesis testing, built into MetaboAnalyst 5.0. b, Pathway enrichment analysis based on human metabolic pathways in the KEGG database quantifies metabolic changes following dox-induced expression of shMTHFD2 or shScramble in OVCAR8 and SKOV3 cell lines. Pathway impact and results of statistical tests using MetaboAnalyst 5.0 are reported from n=5 biological replicates for each condition. c, Differences in metabolites analysed by GC-MS in dox-inducible ovarian cancer lines SKOV3 and OVCAR8 expressing shScramble or shMTHFD2 following treatment with dox (n=5). Violin plots represent data spread, and dashed and dotted lines represent median and quartiles, respectively. Data analysed using Student's two-tailed t-test, corrected for multiple hypothesis testing with the Benjamini-Hochberg method. tRNA, transfer RNA.

FIG. 6 MTHFD2 is a potential collateral lethal target across varied genetic backgrounds and microenvironment pressures. a, UpSet plot of distinguishing and overlapping molecular features (CNA and mutations) of the four ovarian tumour clusters with a 19p13.3 deletion identified by NNMF in FIG. 1d. Rows in the overlap matrix represent all combinations of overlaps among four clusters; vertical bar chart represents number of ovarian samples in each cluster; horizontal bar chart represents number of molecular features specific to each row's overlapping cluster combination; dot plot shows cluster-specific scores of molecular features calculated by NNMF in FIG. 1d. b, Dendrogram estimated from the similarity of molecular features of the four HGSOC clusters with 19p13.3 deletion. c, Multidimensional scaling-based projection of metabolic transcriptional profiles of four HGSOC clusters with 19p13.3 deletion. d, Venn diagram displaying overlap of reactions in reconstructed metabolic models illustrating the distinction between four HGSOC clusters with 19p13.3 deletion. e, Correlation of mRNA expression difference (MTHFD2 versus UQCR11) and dependency scores of MTHFD2 in ovarian cancer lines (Cancer DepMap), estimated using Spearman's rank correlation test (n=37). f, TWI and PAUC scores of top-ranking collateral lethal candidate targets in reconstructed metabolic models of clusters 3, 12, 11 and 32. g, Single-cell gene expression of MTHFD2 and UQCR11 from scRNA-seq data from ascitic fluid of patients with ovarian cancer, coloured by cell type (top), average expression of one-carbon metabolic signature no. 1 (middle) and serine, glycine and threonine metabolic signature no. 1 (bottom). Single-cell expression data were normalized using MAGIC and used to estimate Spearman's correlation coefficient. h, CIBERSORTx-derived, cell-type-specific scores projected on two-dimensional (2D) PHATE embedding of stromal scores representing composition of ovarian tumour samples from TCGA. Joy plots reflect the distribution of cell-specific scores across the quadrants of 2D PHATE embedding. i, Projection of 19p13.3 deletion (log 2(CNA<−0.4)) on the 2D PHATE plane representing the stromal composition of ovarian tumour samples from TCGA. j, Average expression of genes in one-carbon metabolic signature no. 1 across the quadrants of stromal 2D PHATE embedding. k, mRNA expression of MTHFD2 and UQCR11 across the quadrants of the stromal 2D PHATE embedding.

FIG. 7 Remission of UQCR11-null ovarian tumours on collateral lethal targeting of MTHFD2 in vivo. a,b. Schematic for evaluation of in vivo efficacy of collateral lethal targeting of MTHFD2 in mice implanted with subcutaneous tumours from shMTHFD2-expressing OVCAR8 cells (a) or shMTHFD2-expressing SKOV3 cells (b). c,d, Characterization of tumour size and weight from mice subcutaneously implanted with shMTFHD2-expressing OVCAR8 cells (c) or SKOV3 cells (d) and fed either regular food (ctrl) or food+dox (dox). Data presented as mean±s.d. for n=6 mice in each group, and analysed using unpaired two-tailed t-test. e, Validation of dox-induced knockdown on MTFHD2 via immunoblots to measure MTHFD2 protein expression in shMTHFD2-expressing SKOV3-implanted tumours from control and treatment arms. Data representative of two independent experiments. f,g, Representative immunohistochemistry images (f) and quantification (g) of apoptosis marker, cleaved Caspase 3, in tumour sections from mice implanted with shMTHFD2-expressing OVCAR8 cells and fed with ctrl or dox; n=33 imaged sections for ctrl and n=20 for dox. Data presented as mean±s.d. and analysed using two-sided t-test with Welch's correction.

FIG. 8 CLIM workflow integrates multiobjective metabolic flux analysis and machine learning to identify and validate collateral lethal metabolic targets. (a) Definition of Super Pareto, Infeasible Pareto and Blocked Pareto in context of metabolic deletions. (b) Schematic describing the reconstruction of genome-scale metabolic networks for cancers based on metabolic gene expressions and empirically observed extracellular fluxes. Recon 2.2 is used as the input model with all gene-protein-reaction (GPR) relationships. Average expression of cancer cell-lines and tumor samples along with extracellular fluxes are used to reconstruct a contextual model using the iMAT algorithm. Further, automated and manual curation is performed to edit reactions to fix gaps in the model. Final reconstruction is performed by iMAT's extension rxnMILP algorithm. (c) Derivation of Pareto area under the curve (PAUC) to quantify overall metabolic changes across the entire Pareto frontier between deleted- and non-deleted conditions. (d) Derivation of therapeutic window index (TWI) using sensitivity analysis to quantify influence of metabolic fluxes on metabolic objective functions. (e) Decision-making heuristic for selection of collateral lethal candidate targets by analyzing PAUC and TWI scores.

FIG. 9 Discovery of top-ranking collateral lethal candidate pathways by MOMFA within CLIM. (a) Schematic of highlighted metabolic pathways identified as the top-ranking collateral lethal candidate pathways: PSAT, cytosolic MDH, PC, SHMT1, MTHFD1, and PGI. (b) Metabolic fluxes of ETC reactions encoded by UQCR11 (Complex III), NADH reductase (Complex I), and ATP synthase (Complex V) in UQCR11-null and -intact models. Radar plots represent all the Pareto-optimal flux distributions from maximal biomass to maximal ATP maintenance in the counter-clockwise direction. (c) Metabolic fluxes of top-ranking collateral lethal candidate pathways, PSAT, cytosolic MDH, PC, SHMT1, and PGI in UQCR11-null and -intact models. Radar plots represent all the Pareto-optimal flux distributions from maximal biomass to maximal ATP maintenance in the counter-clockwise direction. (d) TWI and log-transformed PAUC of top-25 ranking collateral lethal candidate pathways. High TWI reflects the selective in silico targeting of UQCR11-null cancer cells compared to UQCR11-intact cells upon inhibition of the metabolic pathway. High log[PAUC] values reflect the change in amplitude of fluxes in the metabolic pathway in UQCR11-null cells compared to UQCR11-intact cells, across the Pareto-optimal metabolic objectives. (e) Expanded results of sensitivity analysis of top-ranking collateral lethal candidate pathways used to calculate TWI. Gradual inhibition of flux leads to a detrimental effect to one or both of the metabolic objectives. The differences in the detrimental effect of flux inhibition on UQCR11-null and -intact models are used to estimate the TWI scores. PSAT, phosphoserine aminotransferase; MDH, malate dehydrogenase; PC, pyruvate carboxylase; SHMT1, serine hydroxymethyltransferase; MTHFD1, methylenetetrahydrofolate dehydrogenase; PGI, phosphoglucoisomerase.

FIG. 10 Exploration of collateral lethal targeting of MTHFD2 in parental and doxycycline-inducible knockdown ovarian cancer lines. (a) Copy-number alterations of UQCR11 and MTHFD2 estimated using GISTIC2.0 with the DNA segmentation data of the entire CCLE panel, shown for four ovarian cancer cell-lines selected for in vitro studies. (b) Cell viability assays comparing effect of MTHFD2 knockdown using two shRNAs in UQCR11-intact (SKOV3) and UQCR11-null (OVCAR8, CAOV3, and HeyA8) cell-lines. Data are representative of two independent experiments and presented as mean±s.d, (n=3), and analyzed using ordinary two-way ANOVA with Dunnett's correction for multiple comparisons. (c) mRNA expression of MTHFD2 measured in inducible-knockdown models of OVCAR8 and SKOV3 with and without doxycycline (5 μg/mL). Data is presented as mean±s.d. and combined from N=5 independent experiments for OVCAR8, N=3 independent experiments for SKOV3, and analyzed using ordinary two-way ANOVA with Dunnett's correction for multiple comparisons. (d) Protein expression of MTHFD2 measured in inducible-knockdown model of OVCAR8 and SKOV3 with and without treatment of doxycycline (5 μg/mL). Representative blots from two independent experiments with R-actin used as loading control. (e) Cell viability of dox-inducible shMTHFD2 OVCAR8 and shMTHFD2 SKOV3 lines measured with varying concentrations of doxycycline treatment (0, 1, 2, 5 g/mL). Data are representative of three independent experiments and presented as mean±s.d. (n=3), and analyzed using ordinary two-way ANOVA with Dunnett's correction for multiple comparisons. (f) mRNA expression of UQCR1 ito evaluate knockdown efficiency of stably expressed shUQCR11 in SKOV3 compared to SKOV3 expressing shScramble. Data are representative of three independent experiments and presented as mean s.d. (n=6). Data analyzed using one-way ANOVA with Welch's test for multiple comparisons. (g) Relative viability of SKOV3 cells expressing shScramble or shUQCR11. Data are representative of three independent experiments and presented as mean±s.d. (n=5). Data analyzed using unpaired two-tailed Student's t-test. (h) Live-cell OCR measured using Resipher for isogenic SKOV3 cell-lines expressing shScramble (UQCR11-intact) and two clones of shUQCR11 (UQCR11-null). Data are representative of two independent experiments. Data are presented as mean±s.e.m. (n=11) and analyzed using ordinary one-way ANOVA with Dunnett's correction for multiple comparisons. (i) Basal OCR measured via Seahorse Mito Stress assay for parental ovarian cancer lines, CAOV3, HEYA8 and OVCAR8 (UQCR11-null), relative to basal OCR in SKOV3 (UQCR11-intact). Data are shown as means from N=5 independent experiments, relative to SKOV3. Data presented as mean±s.d. and analyzed using Welch's two-tailed t-test. (j) Total cellular NAD+ and NADH concentrations in parental ovarian cancer cells, SKOV3 (UQCR11-intact), CAOV3, HEYA8 and OVCAR8 (UQCR11-null). Data are representative of two independent experiments and presented as mean±s.d. (n=3) and analyzed using one-way ANOVA with Dunnett's correction for multiple comparisons. (k) Total cellular NAD+ and NADH concentrations in stable knockdown SKOV3 cells expressing shScramble or shUQCR11. Data are representative of two independent experiments and presented as mean±s.d. (n=3) and analyzed using Welch's two-tailed t-test.

FIG. 11 Investigating alternative metabolic pathways to rescue UQCR11-null cells from collateral lethal targeting of MTHFD2. (a) Correlation between NDUFS7 mRNA expression and copy-number alterations of the 19p13.3 locus, represented by UQCR11 copy-number in TCGA OVCA dataset (n=303). Correlations estimated using Spearman's rank correlation test. (b) Gene expression of NDUFS7 measured via qRT-PCR in UQCR11-null lines (OVCAR8, CAOV3, HeyA8) relative to NDUFS7 expression in UQCR11-intact line (SKOV3). Data representative of two independent experiments presented as mean±s.d. (n=5), analyzed using ordinary one-way ANOVA with Dunnett's correction for multiple comparisons. (c) Doseresponse curve for single-agent inhibition in SKOV3 cells by either Complex I inhibitor, rotenone, or MTHFD2 inhibitor. Curve fit using the SynergyFinder tool implementing a LL4 (four-parameter logistic regression) model. Calculation and visualization of two-drug synergy scores and HSA (highest single agent) models to identify combinations with highest synergy using SynergyFinder. Data are representative of two independent experiments. (d) Ectopic expression of AOX (relative to ACTIN mRNA) in HeyA8 cells verified using two primers. Data are representative of two independent experiments and presented as mean±s.e.m (n=3) and analyzed using two-tailed Welch's t-test. (e) Resistance to MTHFD2 inhibition upon introduction of AOX. AOX or vector controls were ectopically expressed in UQCR11-null HeyA8 cell-lines. Viability decrease was measured 72 h after inhibitor treatment relative to vehicle control. Data are representative of two independent experiments and presented as mean±s.e.m. (n=6) and analyzed using ordinary 2-way ANOVA with Sidak's correction for multiple comparisons. (f) Overexpression of UQCR11 in HeyA8 cells. Data are representative of two independent experiments and presented as mean±s.d. (n=4) and analyzed using two-tailed Welch's t-test. (g) Resistance to MTHFD2 inhibition upon overexpression of UQCR11 in UQCR11-null HeyA8 cells. Viability decrease was measured 72 h after inhibitor treatment relative to vehicle control. Data are representative of two independent experiments and presented as mean±s.d. (n=5) and analyzed using two-tailed Welch's t-test.

FIG. 12 Expansion of UQCR11-MTHFD2 collateral lethality and machine-learning based evaluation of targeting MTHFD2 in endometrial cancer. (a) Correlation between MTHFD2 and UQCR11 mRNA expression only for the TCGA uterine corpus endometrial carcinoma (UCEC) (Pan Cancer, 2018) dataset corresponding to the pan-cancer analysis results in FIG. 2i. Correlations estimated using Spearman's rank correlation test. (b) Most significant focal deletions in chromosomes 1-22, identified by GISTIC2.0 within deleted regions for endometrial cancer samples from the TCGA UCEC and uterine carcinosarcoma (UCS) datasets combined (n=584). Correlation of mRNA expression and copy-number alteration of UQCR11 in the same set of samples for which data was available (n=568). Correlations estimated using Spearman's rank correlation test. (c) Two-layer machine learning model trained using 54 mutations observed in endometrial cancers, select gene-expression panel of 112 mitochondrial genes, and fraction genome altered as input. Inputs for both layers are chosen a priori using linear support vector classification (SVC) for recursive feature elimination, and predictive output of 19p13.3 copy-loss from Layer #1 is used as input to Layer #2. Layer #1 is trained with data available for samples from TCGA and GENIE datasets. Layer #2 is trained using samples from the TCGA dataset. 20% is reserved as test data and not used for training either of the two ML layers. (d) Precisionrecall curves for test and training data for Layer #1 of the multi-layer machine learning model that predicts 19p13.3 copy-loss in endometrial cancers. (e) Summary performance metrics and precision-recall curves for training and test data for layer #2 for the multi-layer model that predicts response to MTHFD2 inhibition in endometrial cancers.

FIG. 13 Deuterium tracing to interrogate paralog pathway-mediated metabolic compensations. (a) Stable-isotope tracing map elucidating the transfer of [²H] and enrichment of downstream metabolites from stable-isotope substrates 3-[²H]-glucose (blue), to trace [²H]-NADPH and cytosolic MTHFD1 activity, and 4-[²H]-glucose (orange) to trace [²H]-NADH and mitochondrial MTHFD2 activity. All four ovarian cancer cell-lines were cultured separately in each of the two tracers, and intracellular metabolites were analyzed after 3, 6, and 24 hours of culture with labeled media. Data are presented as mean±s.d. (n=3). (b) Stable-isotope tracing map elucidating the transfer of ²H and enrichment of downstream metabolites from stable-isotope substrate 2,3,3-[2H]-serine (green) to dissect bidirectionality of SHMT2 and MTHFD2 fluxes in mitochondria and SHMT1 and MTHFD1 in cytosol. Intracellular metabolites were analyzed after 3, 6, and 24 hours of culture with labeled media. Data are presented as mean±s.d. (n=3). Data analyzed using ordinary 2-way ANOVA with Dunnett's correction for multiple comparisons.

FIG. 14 Analysis of global metabolomics and nucleotide pathways of ovarian cancer lines with and without UQCR11 deletion. (a) Pathway enrichment analysis using metabolic pathways in the KEGG database comparing UQCR11-intact and -null ovarian cancer lines from the CCLE panel (n=50). Pathway coverage represents fraction of metabolites in the KEGG pathway that have available data. Data analyzed using weighted z-test for pathway analysis built into MetaboAnalyst 5.0. (b) Pathway enrichment analysis on all metabolite concentrations measured in SKOV3 (UQCR11-intact) and OVCAR8, HeyA8 and CAOV3 (UQCR11-null) cells for metabolites involved in nucleotide metabolism. Pathway coverage, that is, percentage of metabolites measured from each pathway are represented by a gradient color-scale. Statistical significance and impact (effect size) of pathway-level differences estimated by MetaboAnalyst Pathway Analysis tool are represented by circle size and the x-axis, respectively. Data analyzed using weighted z-test for pathway analysis built into MetaboAnalyst 5.0. (c) Differential metabolite levels represented using a volcano plot show statistically significant (−log 10P>1.3) and biologically relevant (fold-change>1.5 or <1.5-1). Data points are colored according to the pathway (purine metabolism, pyrimidine metabolism, or other nucleotides/nucleosides). Data are combined from two independent experiments and analyzed using two-sample t-tests corrected for multiple hypothesis testing, built into MetaboAnalyst 5.0. (n=6). (d) Metabolites involved in pyrimidine synthesis and salvage are measured in SKOV3 (UQCR11-intact) and OVCAR8, HeyA8 and CAOV3 (UQCR11-null) cells and overlaid onto the pyrimidine metabolic pathway map. Metabolite levels are scaled to a range [10, 100] to improve visualization. Data are combined from two independent experiments (n=6). (e) Heatmap of purines, pyrimidines, and pathway intermediate concentrations measured in SKOV3 (UQCR11-intact) and OVCAR8 (UQCR11-null) with dox induction of shScramble or shMTHFD2. (f) Differential purines, pyrimidines and pathway intermediates levels represented using a volcano plot in cells treated with MTHFD2 inhibitor vs. vehicle control. Top panel displays fold-change in SKOV3 cells expressing a shScramble and bottom panel displays fold-change in SKOV3 cells expressing shUQCR11. Data are combined from two independent experiments and analyzed using two-sample t-tests corrected for multiple hypothesis testing, built into MetaboAnalyst 5.0 (n=6). (g) K-means clustering (k=4 clusters) of nucleotides measured in samples shown in (e) and (f). K-means clustering performed using MetaboAnalyst 5.0 and visualized with the first two principal components. (h) De novo pyrimidine synthesis demonstrated by extent of [¹³C₃]-UMP enrichment from [¹³C₄]-aspartate in parental ovarian cancer lines, SKOV3 and OVCAR8, cultured with and without MTHFD2 inhibitor. Data shown is [¹³C₃]-UMP enrichment in MTHFD2 inhibitor-treated cells relative to the respective control cells. Data are representative of two independent experiments and presented as mean±s.d. (n=3) and analyzed using two-tailed Welch's t-test.

FIG. 15 Investigating metabolic consequence of Complex III impairment and activity of paralogous pathways other than oxidative MTHFD2. (a) Effect of MTHFD2 inhibitor (10 PM) and uridine (50 μM) supplementation on viability of parental ovarian cancer lines that are UQCR11—intact (SKOV3) or UQCR11-null (HeyA8, OVCAR8, CAOV3). Data are representative of two independent experiments and presented as mean±s.d. (n=6). Data analyzed with one-way ANOVA with Tukey's correction for multiple comparisons. (b) Effect of MTHFD2 inhibitor and uridine supplementation on viability of isogenic SKOV3 lines expressing shScramble or shUQCR11. Data are representative of two independent experiments and presented as mean±s.d. (n=6). Data analyzed with one-way ANOVA with Tukey's correction for multiple comparisons. (c) Viability of SKOV3 and OVCAR8 cells expressing dox-inducible shScramble or shMTHFD2 supplemented with pyruvate. Data are representative of two independent experiments and presented as mean±s.e.m. (n=6) and analyzed using two-tailed Student's t-test. (d) mRNA expression of NNT, IDH2, IDH3A measured via qRT-PCR after knocking down with shNNT, shIDH2, and shIDH3A, relative to scramble shRNA (shScr), verifying knockdown efficiency in SKOV3 and OVCAR8 cell-lines. Data are representative of two independent experiments and presented as mean±s.d. (n=4). Data analyzed using unpaired two-tailed Student's t-test. (e) Viability of SKOV3 and OVCAR8 cells after transfection of shNNT, shIDH2, and shIDH3A, relative to respective SKOV3 and OVCAR8 controls transfected with scramble shRNA (shScr). Data are representative of two independent experiments and presented as mean±s.d. (n=5). Data are analyzed using ordinary one-way ANOVA with multiple comparisons. (f) SKOV3 and OVCAR8 cells transfected with shScramble, shNNT, shIDH2, and shIDH3A treated with MTHFD2 inhibitor (10 μM) for 72 h. Loss of viability calculated by comparing respective viability in vehicle control-treated cells to MTHFD2 inhibitor-treated cells. Data are representative of two independent experiments and presented as mean±s.e.m. (n=6) and analyzed using ordinary one-way ANOVA with Dunnett's correction for multiple comparisons. Error bars are calculated using propagation of error principles.

FIG. 16 Targeting MTHFD2 across co-occurring genetic mutations and heterogenous stromal microenvironment in tumors. (a) Multidimensional scaling-based 2D projection of complete transcriptional profiles of the four HGSOC clusters with 19p13.3 deletion. (b) Venn diagram representing the overlap of reactions in reconstruction of metabolic models illustrating the average expression of four ovarian tumor clusters with 19p13.3 deletion and the overall original ovarian cancer model with 19p13.3 deletion used in FIG. 2. (c) Unsupervised PHATE analysis on scRNASeq data from ascites of ovarian cancer patients to assign cell-types. (d) UQCR11 and MTHFD2 mRNA expression across cell types projected on 2D PHATE embedding of scRNASeq data. (e-g) Average expression (pathway score) of genes in the one carbon metabolic signature #1 (e), serine glycine threonine metabolism signature #1 (f), and the NAD+ synthesis metabolism (g) in ovarian tumors from TCGA with 19p13.3 intact or 19p13.3 deletion. Data are represented as mean±s.d, analyzed using Student's two-tailed t-test with Welch's correction (e-g). (h) Average expression (pathway score) of genes remaining signatures related to the serine-glycine one carbon metabolism and mitochondrial metabolism across the quadrants of the stromal 2D PHATE embedding. (i) UQCR11 mRNA expression and one carbon metabolism (signature #1) pathway score in microdissected malignant and stromal ovarian tumor compartments (GSE115635). Pairs that have reduced UQCR11 expression in malignant compartments compared to stromal compartments (lower than median of expression difference) are colored red, while those with higher UQCR11 expression in malignant compartments are marked in grey. Data analyzed using paired two-tailed Student's t-test (n=17 red pairs, n=17 grey pairs).

### DETAILED DESCRIPTION

Recurrent loss-of-function deletions cause frequent inactivation of tumour suppressor genes but often also involve the collateral deletion of essential genes in chromosomal proximity, engendering dependence on paralogues that maintain similar function. Although these paralogues are attractive anticancer targets, no methodology exists to uncover such collateral lethal genes. Here we provide a framework for collateral lethal gene identification via metabolic fluxes, CLIM, and use it to reveal MTHFD2 as a collateral lethal gene in UQCR11-deleted ovarian tumours. We show that MTHFD2 has a non-canonical oxidative function to provide mitochondrial NAD+, and demonstrate the regulation of systemic metabolic activity by the paralogue metabolic pathway maintaining metabolic flux compensation. This UQCR11-MTHFD2 collateral lethality is confirmed in vivo, with MTHFD2 inhibition leading to complete remission of UQCR11-deleted ovarian tumours. Using CLIM's machine learning and genome-scale metabolic flux analysis, we elucidate the broad efficacy of targeting MTHFD2 despite distinct cancer genetic profiles co-occurring with UQCR11 deletion and irrespective of stromal compositions of tumours.

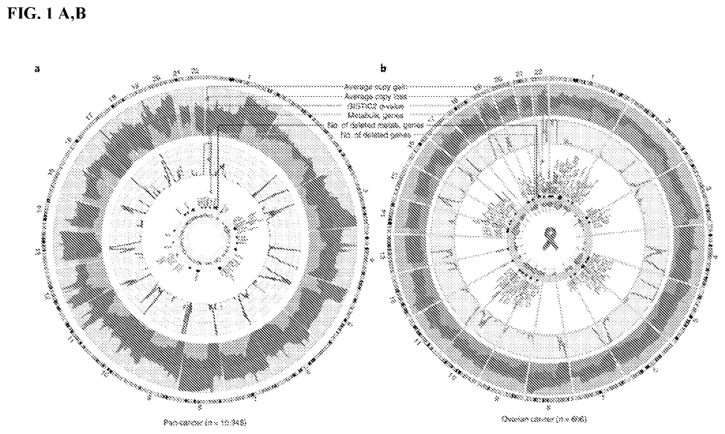

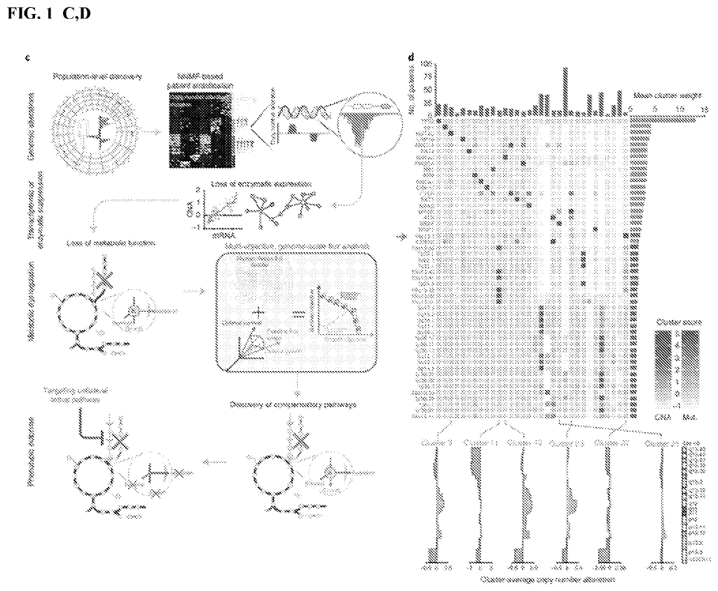

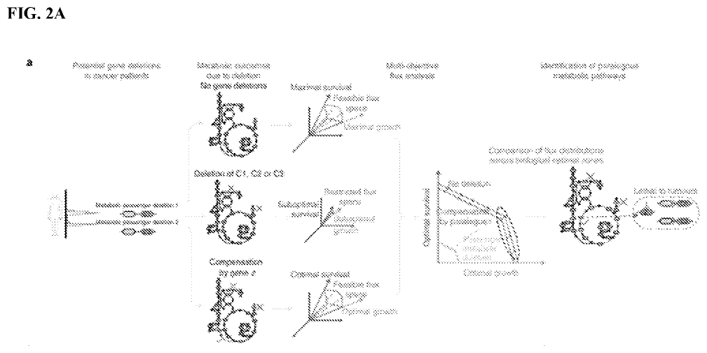

We have developed an approach, defined as collateral lethal genes identification via metabolic fluxes (CLIM), that identifies personalized metabolic targets in cancers with distinct mutational signatures using a combination of data mining, machine learning and multi-objective metabolic flux analysis techniques. To determine putative frequent heterozygous and homozygous deletions of metabolic enzyme-encoding genes at the global genomic landscape level, we analysed copy number alteration (CNA) data from The Cancer Genome Atlas (TCGA) pan-cancer dataset of >10,000 patients with GISTIC2.0 (ref 14) and uncovered the presence of 80 frequently deleted chromosomal regions (FIG. 1a). Despite, the genetic heterogeneity of tumours from 32 sites of origin, these data strongly indicate that certain genomic loss events are common among cancer alterations and are conserved across primary sites¹⁴. Interestingly these focal deletions contain at least 49 metabolic genes, which further supports our hypothesis of prevalent loss of metabolic genes in cancers (FIG. 1a). By performing a similar analysis on CNA from tumours categorized by the site of origin, we revealed a similar pattern of frequently occurring chromosomal deletions containing metabolic genes. Solid tumours showed higher numbers of chromosomal deletions compared with acute myeloid leukaemia (AML) samples, and the number of potentially lost metabolic genes ranged from 26 in AML to 212 in invasive breast carcinoma. Interestingly, cross-referencing these metabolic genes to a database of genetic paralogues uncovered that almost half of the deleted metabolic genes did not have genetic paralogues. This further highlighted the need to identify potential collateral lethal pathways in tumour metabolic networks. Further, from the high-grade serous ovarian carcinoma (HGSOC) TCGA dataset with prevalent chromosomal alterations we observed 60 frequent deletions containing >170 metabolic genes (FIG. 1b). We devised an integrated workflow that utilizes genomic and transcriptomic profiles of cancer patients to identify metabolic deletions, reconstructs genome-scale metabolic models (GSMMs), performs cellular objectives-based metabolic flux analysis and uncovers compensatory metabolic pathways for collateral lethal targeting (FIG. 1c). Interestingly, the most prevalent deletion on the 19p13.3 locus in HGSOC was identified not only by the GISTIC2.0 algorithm but also by our machine learning approach, which stratifies patients based on oncogenic mutations and CNA (FIG. 1c,d). The 19p13.3-deleted region contains the tumour suppressor LKB1, which is mutated or deleted in Peutz-Jeghers syndrome and several cancers and is involved in the tumorigenesis of cervical and ovarian cancers^{15,16} with homozygous loss in 5% and heterozygous loss in up to 30% of HGSOC tumours from TCGA¹⁷. To delineate patient clusters with distinguishable chromosomal deletions and mutations at a population level on the HGSOC dataset, we performed unsupervised clustering analysis based on molecular features using non-negative matrix factorization (NNMF). Putative oncogenic mutations had the highest scores in defining NNMF clusters; remarkably, 19p13.3 was the CNA among the cluster-defining molecular feature (FIG. 1d). 19p13.3 deletion was a distinguishing feature for five NNMF clusters, and contrasting the average CNA for these patient clusters (clusters 3, 11, 12, 23 and 32) showed consistent copy loss in 19p13.3 whereas other loci of chromosome 19 had no discernible commonalities. The depth of 19p13.3 deletion in these clusters is further highlighted when comparing the CNA of these five clusters with that of cluster 21, which had insignificant CNA of the 19p13.3 locus. Ubiquinol-cytochrome c reductase, complex III subunit XI (UQCR11), a gene that encodes a subunit of complex III of the electron-transport chain (ETC), lies within this locus. Furthermore, copy loss of UQCR11 strongly correlates with reduced messenger RNA expression of UQCR11. Because UQCR11 has no known genetic paralogue, its loss would indicate mitochondrial energy metabolism dysfunction. Although the systemic pathological role of complex III dysfunction in healthy cells is rare and undercharacterized¹⁸, it has been shown to impair oxidative phosphorylation (OXPHOS) in breast cancer^{19,20} and contributes to destabilization of complex I²¹. Because OXPHOS is an essential source of ATP for rapidly proliferating cancer cells, mitochondrial dysfunction drives metabolic rewiring to meet high bioenergetic demands^{22,23}.

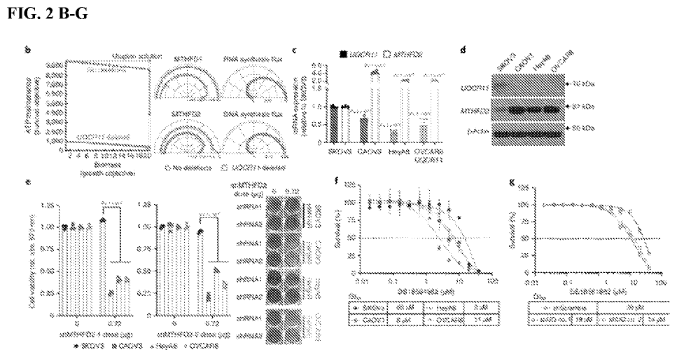

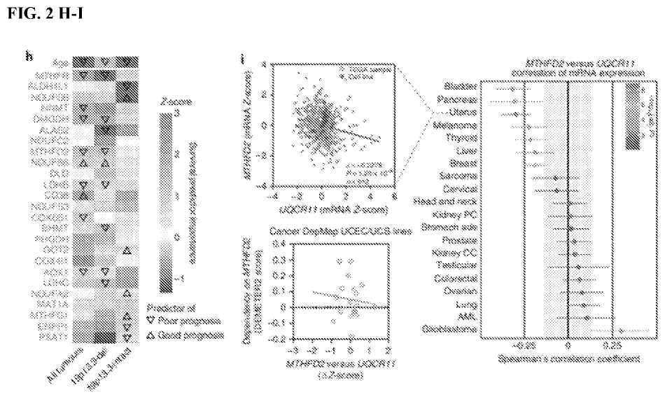

This led us to hypothesize that ovarian cancer cells with the 19p13.3 deletion and ETC impairment would shift their reliance onto other metabolic pathways to maintain their energy requirements and NAD+/NADH balance, revealing a collateral lethal metabolic target specific to UQCR11-null ovarian cancer. To capture the plasticity of cancer metabolism, we relied on comprehensive GSMMs to dissect the effect of UQCR11 loss and ETC impairment in HGSOC. Multiple cellular objectives that compete for metabolic resources reflect the adaptability of cancer cells to varied intrinsic and extrinsic pressures. Herein, we selected competing objectives of growth (biomass production) and survival (ATP production). We employed our multi-objective metabolic flux analysis (MOMFA) algorithm on reconstructed GSMM for 19p13.3-deleted ovarian cancers to reveal the compensatory metabolic pathways employed by cancer cells after loss of UQCR11-mediated reactions (FIG. 2a,b). Those compensatory pathways that were ubiquitously active across the spectrum of adaptability were considered as viable candidates. From these, pathways that detrimentally affected 19p13.3-deleted cells when inhibited in silico while also having a minimal effect on cells without 19p13.3 deletions were ranked higher by CLIM. The top chosen candidate pathways catalyzed by enzymes encoded by methylenetetrahydrofolate dehydrogenase, MTHFD1/2 and phosphoserine aminotransferase PSAT-revealed an intriguing rewiring in cells with UQCR11 deletion. Surprisingly, despite significant rewiring of mitochondrial one-carbon metabolism, the predicted nucleotide synthesis fluxes were minimally affected (FIG. 2b). MTHFD2 was predicted as the most critical component of one-carbon metabolism and emerged as a paralogous pathway that is contextually active in cells with UQCR11 deletion. Whole-genome sequencing data from the Cancer Cell Line Encyclopedia (CCLE) helped identify four ovarian cancer lines that would represent copy loss and diploid UQCR11. CAOV3, HEYA8 and OVCAR8 with heterozygous UQCR11 deletion (hereafter termed UQRC11-null) and SKOV3 with diploid UQCR11 (hereafter termed UQCR11-intact) were employed for evaluation of collateral lethal targeting in vitro. SKOV3 expressed UQCR11 at the transcript and protein levels while CAOV3, HeyA8 and OVCAR8 had undetectable UQCR11 protein expression (FIG. 2c,d), validating our in vitro models. MTHFD2 short hairpin RNA transfection decreased cell viability in a dose-dependent manner in UQCR11-null OVCAR8, CAOV3 and HeyA8 cell lines but had no effect on the UQCR11-intact SKOV3 line (FIG. 2e). These observations were corroborated with doxycycline (dox)-inducible shRNA-mediated stable knockdown of MTHFD2 in UQCR11-intact SKOV3 and UQCR11-null OVCAR8 lines. Specifically, dox induction of shMTHFD2 decreased the cell viability of OVCAR8 but had no effect on SKOV3 cells. This collateral lethal targeting effect was also confirmed using DS18561882, a highly potent and selective MTHFD2 inhibitor²⁴ (FIG. 2f), with UQCR11-null cells showing six- to 20-fold lower concentration causing 50% cell growth inhibition (GI50) compared with UQCR11-intact cells. Furthermore, in an isogenic model developed by stably expressing shUQCR11 in UQCR11-intact SKOV3 cells, GISO concentrations were significantly reduced in SKOV3 cells with UQCR11 knocked down, indicating a strong functional collateral lethal link between UQCR11 deletion and targeting MTHFD2 (FIG. 2g). To discover the metabolic link between UQCR11 and MTHFD2, we first assessed the metabolic influence of UQCR11 deletion on ovarian cancer cells. Notably, UQCR11 knockdown did not affect cell viability but significantly reduced total cellular oxygen consumption rate (OCR). Further, basal OCR in UQCR11-null parental ovarian cancer lines was reduced compared with UQCR11-intact cells. At the metabolic level, NAD+ levels were significantly lower and NADH levels trended lower in UQCR11-null cells compared with UQCR11-intact, in both parental and isogenic systems. Furthermore, ETC impairment in UQCR11-null tumours was possibly exacerbated by the loss of NDUFS7, which lies in the same locus as UQCR11. To recapitulate a functional impairment of complex I, we inhibited its activity in UQCR11-intact SKOV3 cells with the complex I inhibitor, rotenone, and treated them with MTHFD2 inhibitor. Notably, pharmacological co-inhibition of complex I and MTHFD2 had a synergistic effect on the viability of SKOV3 cells, indicating the existence of a collateral lethal link between ETC impairment and MTHFD2 activity. Additionally, re-establishing complex III activity in UQCR11-null HeyA8 cells by ectopically expressing alternative oxidase, AOX, from Ciona intestinalis²⁵⁻²⁷ and overexpressing UQCR11 partially rescued these cells from MTH1FD2 inhibitor-mediated cell death. These observations suggested that the lethality of targeting MTHFD2 in UQCR11-null cells was linked to ETC impairment, potentially via the imbalance of NAD+. To establish the clinical relevance of MTHFD2 collateral lethality, random survival forest (RSF) models were trained for prediction of prognosis based on age and gene expression of metabolic pathways related to one-carbon metabolism, ETC and the Krebs (TCA) cycle. As expected, age best predicted mortality in all cohorts. Remarkably, MTHFD2 and MTHFR of one-carbon metabolism predicted poor prognosis in the 1⁹p13.3-deleted cohort but not in the 19p13.3-intact cohort (FIG. 2h). These unsupervised machine learning methods predicting survival, therefore, provided further evidence on the importance of MTHFD2 and related metabolic genes in UQCR11-null ovarian tumours. We also sought to determine whether MTHFD2-centric rewiring occurs in other cancers with UQCR11 deletion. Remarkably, the strong negative correlation between UQCR11 and MTHFD2 transcripts indicated potential collateral lethal pairing in many cancers across the TCGA Pan-Cancer cohort (FIG. 2i); only gliomas showed a positive correlation between MTHFD2 and UQCR11. Interestingly, gliomas are outliers compared with other cancers because low MTHFD2 expression is correlated with poor prognosis, alluding to MTHFD2 dependence unrelated to the col-lateral lethal pairing with UQCR11 (refs. 28,29). Notably, uterine cancers have a strong negative correlation, as seen in combined TCGA datasets for uterine corpus endometrial carcinoma (UCEC) and uterine carcinosarcoma (UCS), and uterine cancer lines from the CCLE (FIG. 2i, top inset). This correlation was strongest within UCEC samples. In addition, deletion of the 19p13.3 locus was independently identified as significant in UCEC tumours using GISTIC (q-value=2.3×10-21).

This deletion is notable given that previous observations in mouse models revealed that loss of LKB1 is linked to UCEC pro-gression^{30,31} Furthermore, the consensus score of MTHFD2 dependency in endometrial cancer lines reported in the Cancer DepMap portal³² displayed a correlation that trended negatively with the ratio of MTHFD2 to UQCR11 expression, indicating MTHFD2 dependency in UCEC with low UQCR11 (FIG. 2i, bottom inset). Furthermore, we see that patients with uterine cancer and UQCR11 copy loss have a significantly poorer prognosis compared with those with diploid or amplified UQCR11 (FIG. 2j, yellow versus red and blue). Their poor outcomes are further exacerbated when tumours have both UQCR11 copy loss and high MTHFD2 gene expression compared with those patients with low MTHFD2 expression (FIG. 2j, red versus blue). Similarly, patients with tumours showing low UQCR11 gene expression and high MTHFD2 expression have poorer outcomes compared with patients with low expression of both UQCR11 and MTHFD2 (FIG. 2k), suggesting that tumours with 19p13.3 deletion but higher expression of MTHFD2 are more aggressive compared with those with lower MTHFD2 expression. Notably, a multilayer machine learning model within CLIM could predict UQCR11 copy loss and response to MTHFD2 inhibition in UCEC cancers with minimal mutation and gene expression data. Interestingly, UCEC tumours with a TP53 mutation but not a PTEN mutation are predicted to display 19p13.3 loss whereas tumours with a TP53 and PTEN mutation, or no TP53 mutation, do not. These data highlight that collateral lethal targets discovered by CLIM can capture targets across cancer types based on mutational and transcriptional information.

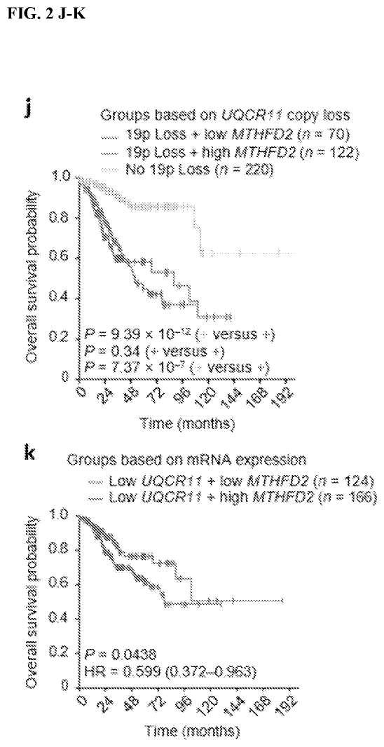

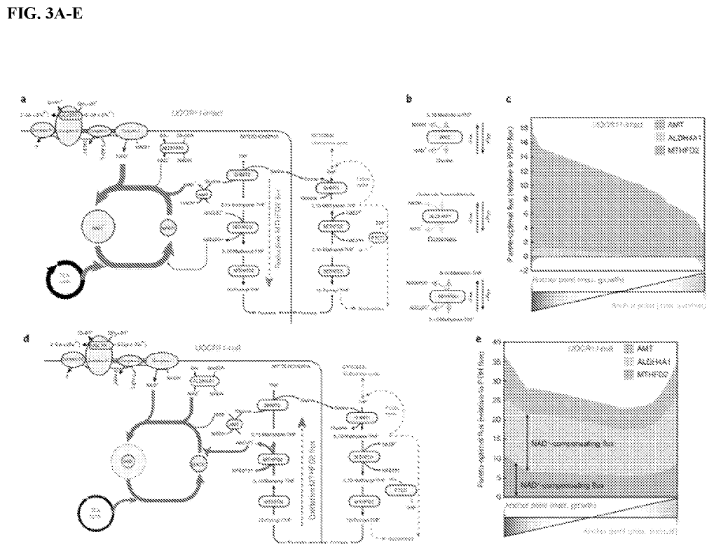

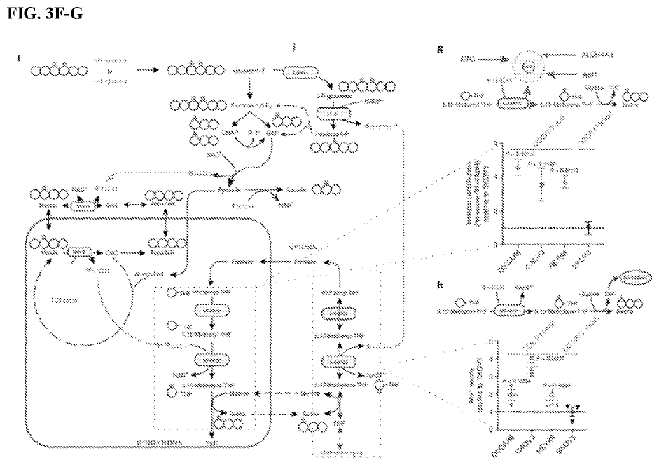

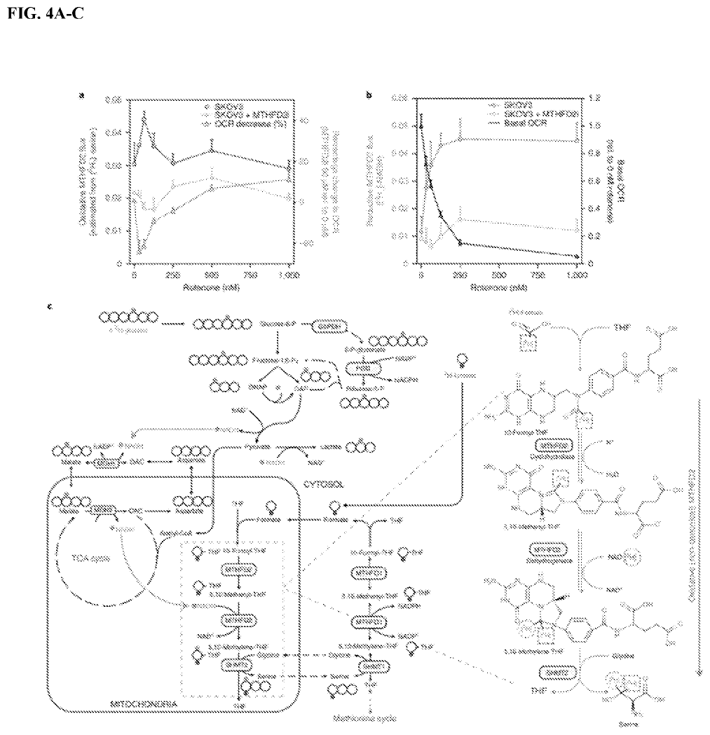

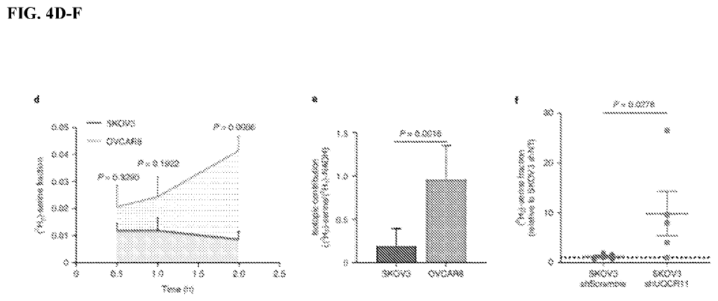

Although collateral lethal gene identification and its therapeutic targeting to control tumour growth have previously been demonstrated^{2,4,33,34}, the regulation of systemic metabolic activity by paralogous metabolic pathways to achieve metabolic flux compensation has been overlooked. CLIM showed the unique behavior of the mitochondrial MTHFD2 pathway by elucidating its reversed direction, producing NAD(P)+ with respect to the widely observed NAD(P)H− producing flux of mitochondrial SHMT2 and MTHFD2 reactions³⁵⁻³⁷. The MOMFA algorithm within the CLIM workflow indicated that this was due to the unique pleiotropic properties of MTHFD2 and its ability to generate NAD+ (FIG. 2b). We established the existence of metabolic compensation using isotope tracing with 2H deuterium-labelled substrates and validated MOMFA-predicted fluxes. Although the ETC substantively contributes to maintaining the NAD+/NADH ratio in mitochondria, three other pathways can also oxidize NADH to NAD+: aminomethyltransferases (AMT), glutamate γ-semialdehyde dehydrogenase (ALDH4A1) and MTHFD2 (FIG. 3a,b). In UQCR11-intact cells MOMFA predicted that, apart from ETC, AMT was the only pathway that would oxidize NADH to NAD+ across the spectrum of metabolic adaptations possible, represented by competing cellular objectives of growth and survival (FIG. 3a-c (teal areas)). MTHFD2 and ALDH4A1 fluxes contributed minimally to maintenance of mitochondrial NAD+ pools (FIG. 3b,c, blue and pink areas); in fact, net MTHFD2 flux was responsible for NAD(P)+ reduction to NAD(P)H (FIG. 3b,c, pink area). In contrast, ALDH4A1 fluxes were higher and AMT fluxes lower across the metabolic states between growth and survival in UQCR11-null cells compared with UQCR11-intact (FIG. 3b, d, e, teal and blue areas). The most remarkable change, however, was MTHFD2 flux, which compensated for UQCR11 deletion-induced NAD+ depletion by maintaining a net flux oxidizing NAD(P)H to NAD(P)+ across all possible optimal metabolic states between the two extreme cellular objectives (FIG. 3b,d, e, pink areas). This non-canonical flux of MTHFD2 (hereby referred to as oxidative MTHFD2 flux) was not observed in UQCR11-intact cells in silico, and further strengthened the candidacy of MTHFD2 as a collateral lethal target in UQCR11-null cells. Regulation of one-carbon metabolism, including MTHFD2, has been studied extensively in the context of regulation of cofactors and NAD(P)H;³⁸⁻⁴¹ however, contextual directionality in the MTHFD2 pathway has not been reported. Parallel tracer experiments with three [2H]-labelled substrates (3-[2H]-glucose, 4-[2H]-glucose and 2,3,3-[2H]-serine) allowed uncovering of mechanistic underpinnings in this pathway in vitro. Labelling of cytosolic NADH from 4-[2H]-glucose in all four cell lines is evident from the labelled lactate. Further, reaction catalyzed by cytosolic malate dehydrogenase (MDH1) transfers [2H] from NADH to cytosolic malate, which is transported to the mitochondria where malate transfers [2H] back to NADH (FIG. 3f)³⁶. Enrichment of total cellular malate indicates that there is indeed a transfer of [2H] between malate and NADH^{36,42}. Only oxidative MTHFD2 flux can transfer [2H] from mitochondrial NADH to 5,1 O-methylene-THF, which is subsequently transferred from 5,10-methylene-THF to mitochondrial serine by SHMT2 (FIG. 3f). Remarkably, the isotopic enrichment ratio of [2H]-serine to [2H]-NADH, which represents this oxidative MTHFD2 flux, was found to be threefold higher in UQCR11-null cells compared with UQCR11-intact cells (FIG. 3f,g). The distinct labelling of [2H]-serine from [2H]-NADH via oxidative MTHFD flux is possible only through mitochondrial MTHFD2 activity, which oxidizes NAD(P)H, while cytosolic MTHFD1 can use only NADPH. These data provide the first strong piece of evidence that MTHFD2 flux is reversed during ETC impairment, as in UQCR11-null cells. Furthermore, tracing with 2,3,3-[2H]-serine corroborated that flux through reversible MTHFD2 and SHMT2 is dominantly oxidative. Enrichment of M+1 serine increased over time in UQCR11-null cells and concomitantly decreased in UQCR11-intact cells. This, accompanied by decrease in M+3 serine and M+1 glycine, was possible only when MTHFD2 reduced 5,10-methenyl-THF to 5,10-methylene-THF while producing NAD(P)+, followed by demethylation of 5,10-methylene-THF to serine from glycine by SHMT2 in the direction of oxidative MTHFD2. From 3-[2H]-glucose tracer experiments, we followed the labelling of [2H]-NADPH in the cytosol to serine via MTHFD1, a prerequisite for nucleotide synthesis. Because enrichment of serine was similar across UQCR11-null and -intact cells, we concluded that nucleotide synthesis maintained by MTHFD1 was unaffected by UQCR11 deletion (FIG. 3f,h). This was also predicted by MOMFA, where the net fluxes of DNA and RNA synthesis were similar between UQCR11-null and -intact conditions (FIG. 2b). Importantly, UQCR11 copy loss potentially leads to hypomorphs where mitochondrial respiration is partially impaired, as observed from parental and UQCR11 knockdowns that survive despite lower OCR and reducing factors. To dissect the emergence of oxidation of NADH through MTHFD2 during ETC inhibition, we treated UQCR11-intact SKOV3 cells with increasing doses of rotenone. Specifically, oxidative MTHFD2 flux first increased with increasing, yet low, rotenone doses but returned to original levels as doses were further increased (FIG. 4a). This flux is suppressed when cells are treated with the MTHFD2 inhibitor (FIG. 4a). Notably, in the low range of rotenone doses where oxidative MTHFD2 flux increased, there was a synergistic decrease in OCR in SKOV3 cells treated with rotenone and MTHFD2 inhibitor (FIG. 4a). At doses >62 nM, MTHFD2 inhibition did not detrimentally affect OCR despite a monotonic decrease in basal OCR with increasing rotenone concentrations (FIG. 4a,b). In the case of reductive MTHFD2 flux there was a modest increase at low rotenone doses, which became most pronounced at the highest doses (FIG. 4b). Expectedly, treatment with the MTHFD2 inhibitor greatly reduced reductive MTHFD2 flux (FIG. 4b). Cumulatively, these data support the emergence of oxidative MTHFD2 flux as a compensatory pathway for NAD+ production in cells with mild ETC impairment. This phenomenon subsequently yielded to reductive MTHFD2 flux under strong ETC inhibition, as previously demonstrated⁴⁰.

Next, we employed a combination tracer experiment with 4-[2H]-glucose and [2H]-formate to further deconvolute oxidative mitochondrial MTHFD2 activity (FIG. 4c). Early time-course measurements ensured that serine enrichment from background [2H] labelling was minimized and remained specific to the oxidative flux of mitochondrial MTHFD2. We observed that enrichment of [2H2]-serine monotonically increased in OVCAR8 cells in the first 120 min of tracer introduction and was significantly higher at 120 min than in SKOV3 cells in which enrichment remained constant (FIG. 4d). Remarkably, the normalized contribution of [2H] from [2H]-NADH to [2H2]-serine, representing relative oxidative MTHFD2 flux, was significantly higher in OVCAR8 cells compared with SKOV3 cells (FIG. 4e). There was also significantly higher enrichment of serine in isogenic SKOV3 cells expressing shUQCR11, demonstrating the emergence of oxidative MTHFD2 flux when ETC is impaired (FIG. 4f).

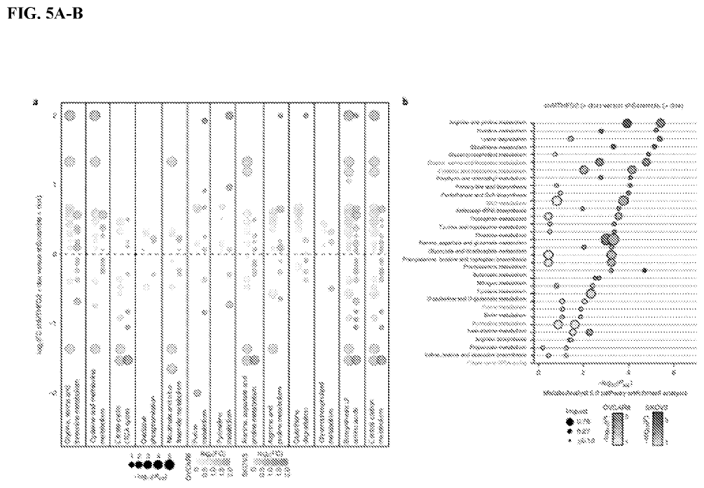

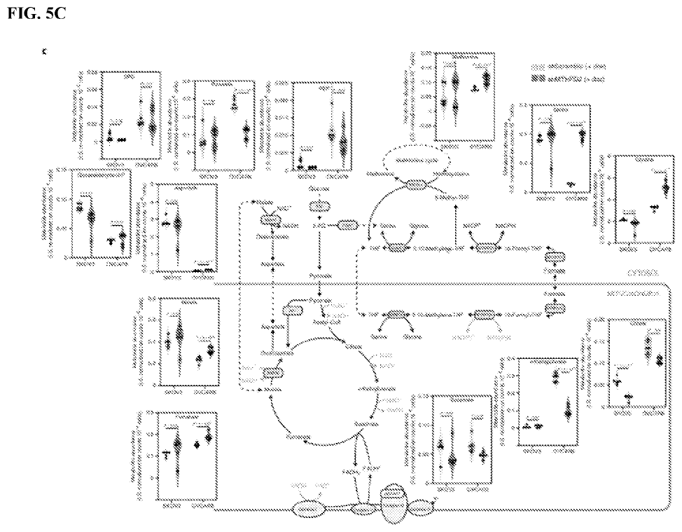

We performed global metabolomics profiling to investigate systemic metabolic changes when MTHFD2 was knocked down in UQCR11-null and -intact cells (FIG. 5a). We observed differential levels of metabolites across central carbon, amino acid, purine and pyrimidine metabolism in both UQCR11-null (OVCAR8) and -intact (SKOV3) cells. However, changes in metabolites involved in serine-glycine one-carbon metabolism encompassing MTHFD2 reactions, the methionine cycle, the TCA cycle, OXPHOS and NAD+ metabolism were more pronounced in OVCAR8 cells compared with SKOV3 cells following MTHFD2 knockdown (FIG. 5a). This highlighted the selective reliance of UQCR11-null cells on MTHFD2 and one-carbon metabolic pathways. Further, metabolite set enrichment analysis elucidated differences in the same pathways that were more pronounced in UQCR11-null cells compared with -intact cells following MTHFD2 knockdown (FIG. 5b). Notably, serine, glycine and methionine accumulated in OVCAR8 following MTHFD2 knockdown, signaling a reduction in serine oxidation and a disruption in the coupled folate and methionine cycles, whereas levels of these metabolites were largely unaffected by MTHFD2 knockdown in SKOV3 cells (FIG. 5c). Furthermore, in OVCAR8 cells levels of TCA intermediates citrate, α-ketoglutarate and succinate were reduced whereas fumarate, malate and aspartate were increased following MTHFD2 knockdown (FIG. 5c). These data indicated that TCA cycle reactions upstream of succinate dehydrogenase (complex II) and complex III in the ETC that rely on NAD+/NADH as cofactors were affected due to imbalance of NAD+/NADH within the mitochondria when MTHFD2 was targeted in OVCAR8 cells but not in SKOV3 cells (FIG. 5c). Additionally, MTHFD2 knockdown had no effect on glycolytic intermediates in either OVCAR8 or SKOV3 cells (FIG. 5c), highlighting its targeted influence on mitochondrial metabolism of UQCR11-null cells. Metabolomics profiles of UQCR11-null and -intact ovarian cancer lines from the CCLE panel also show distinction in serine-glycine one-carbon metabolism and its related methionine cycle pathways⁴⁵. Previous reports demonstrated that complex III impairment impedes pyrimidine synthesis and shifts its reliance onto uridine and pyruvate, which warranted investigation of nucleotide profiles of UQCR11-null and -intact cells^{46,47}. We profiled a panel of >100 nucleotides and nucleotide intermediates involved in de novo synthesis, salvage and degradation pathways in parental ovarian cancer lines. Enrichment analysis revealed that synthesis of purine, rather than pyrimidine, in UQCR11-null cells was significantly different compared with that in UQCR11-intact cells, in fact, out of ten statistically significant (adjusted P<0.05) and biologically relevant (fold-change>1.5 or <1/1.5), seven are purine intermediates but only two are pyrimidine intermediates. Remarkably, nucleotide profiling showed that MTHFD2 inhibition affected only a small subset of nucleotides in isogenic SKOV3 models, with the most marked changes in SKOV3 shScramble cells (ten out 49 differentially abundant metabolites) compared with SKOV3 shUQCR11 (three out of 49). K-means clustering of metabolite levels measured in these four groups separated into four clusters revealed a distinction between SKOV3 shScramble cells treated with or without MTHFD2 inhibitor; however, SKOV3 shUQCR11 cells fall within the same cluster regardless of MTHFD2 inhibitor treatment. Notably, these data support that pyrimidine synthesis is sustained during ETC disruption⁴⁸ and that these cells maintain pyrimidine levels even when UQCR11 is deficient. This is presumably due to partial ETC impairment, with UQCR11 expression being sufficient to support dihydro-orotate dehydrogenase activity. Notably, de novo pyrimidine synthesis flux measured using [¹³C₄]-aspartate tracer was similarly reduced in SKOV3 and OVCAR8 cells following MTHFD2 inhibition. Analysis of the gene expression of distinct pyrimidine synthesis pathways in TCGA ovarian tumours also showed no down-regulation in UQCR11-null tumours compared with UQCR11-intact. MTHFD2 inhibitor suppressed proliferation of both UQCR11-null parental and SKOV3 shUQCR11 cells compared with UQCR11-intact cells; however, uridine was unable to rescue cells from MTHFD2 inhibition. Similarly, pyruvate could not rescue UQCR11-null cells when MTHFD2 was knocked down in SKOV3 and OVCAR8 cells. The limited extent of UQCR11 loss in 19p13.3-deleted ovarian tumours did not induce a complex III-dependent reduction in de novo pyrimidine synthesis. Further, MTHFD2 inhibition had a widespread effect on nucleotide metabolism in UQCR11-intact cells, probably due to reliance on reductive MTHFD2. However, its direct effect on pyrimidine synthesis was not exacerbated in UQCR11-null cells. Cumulatively, this suggested that inhibition of MTHFD2 had disrupted serine-glycine one-carbon metabolism as seen in FIG. 4, consequently affecting purine synthesis.

Together, dynamic tracing and metabolomics data provided evidence that the collateral lethal gene pairing of UQCR11 and MTHFD2 maintained metabolic compensation via the intricate rewiring of compartmentalized mitochondrial NAD+-producing fluxes. We developed isogenic SKOV3 and OVCAR8 cell lines with NNT, IDH2 or IDH3A knocked down to investigate whether reversible TCA cycle enzymes can act as paralogous NAD+ sources⁴⁹. Importantly, knocking down these genes did not reduce SKOV3 viability. Interestingly, in UQCR11-null cells, knocking down NNT or IDH3A, both of which can be reversed to produce NAD+, led to a modest 15% reduction in viability compared with the 50% reduction observed with MTHFD2 knockdown (FIG. 2e). To understand whether these reactions could also supply NAD+ in UQCR11-null cells, we investigated whether their suppression would synergize with MTHFD2 inhibition. Remarkably, the effect of MTHFD2 inhibition was not influenced by these knockdowns, in either UQCR11-intact or -null cells. These empirical observations agreed with the MOMFA algorithm, which predicted oxidative MTHFD2 as a primary collateral lethal target but not IDH or NNT. This highlighted the unique ability of CLM to predict collateral lethal metabolic target pairs that have compensatory metabolic links elucidated via metabolic flux analysis and validated by empirical stable-isotope tracing.

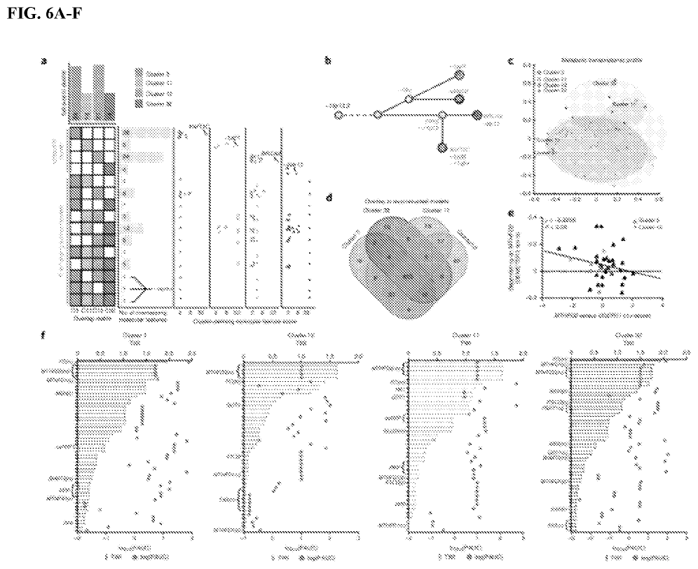

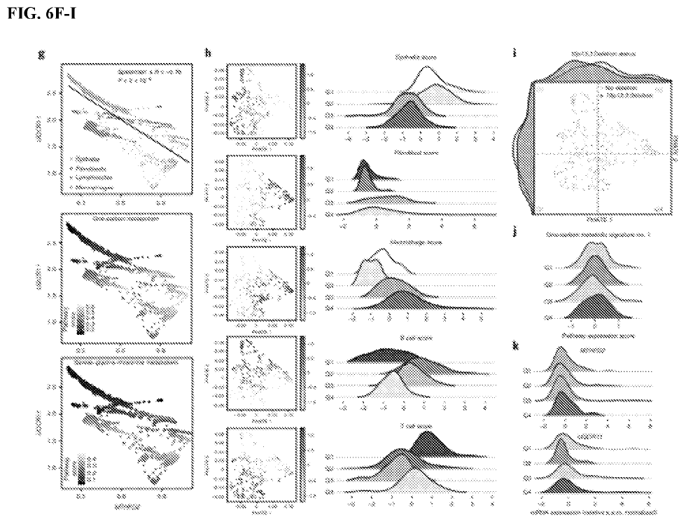

MTHFD2 was predicted and validated as a collateral lethal target in ovarian tumours with 19p13.3 deletion (FIGS. 1 and 2). However, distinct genetic backgrounds observed in ovarian tumours can potentially influence their systemic metabolic reprogramming. We looked at those NNMF-stratified tumours (FIG. 1d) with distinct genetic backgrounds to discern whether mutations co-occurring with 19p13.3 loss could confound the collateral lethal effect of targeting MTHFD2. We selected clusters 3, 11, 12 and 32, which had the 19p13.3 deletion as the only overlapping molecular feature, and 28, 5, 24 and 4 unique molecular features, respectively (FIG. 6a). KMT2C and BRCA2 mutations were unique to clusters 3 and 12, respectively, but TP53 mutation and 11p15 deletion were common to both (FIG. 6a,b). Deletions in 1p21 and 22q13 loci were the distinguishing features for clusters 11 and 32, respectively, along with underlying 19q deletion (FIG. 6a,b). Although the dimensionally reduced projections of complete transcriptional profiles were indistinguishable, the metabolic transcriptional profile showed differences between clusters 11 and 32, and their distinction from clusters 3 and 12, which were indistinguishable from each other (FIG. 6c). Notably, the reconstructed GSMMs for the four clusters also displayed different reactions akin to their metabolic transcriptional profiles (FIG. 6d). Importantly, most of the reactions (413 out of 620) were common across all reconstructed models for the four clusters and the average ovarian cancer model, suggesting the existence of a core metabolic subnetwork unaffected by genetic heterogeneity (FIG. 6d). We investigated the transcriptomic dissimilarities of these clusters and their influence on the utility of MTHFD2 as a collateral lethal target at a phenotypic level. Comparing the dependency score of MTHFD2 knockdown in ovarian cancer cell lines, as evaluated by the Cancer Dependency Map RNA interference screens, revealed that the correlation between MTHFD2 essentiality and MTHFD2-to-UQCR11 expression difference trended negatively (FIG. 6e). This indicated that higher MTHFD2 and lower UQCR11 expression predicted a higher dependence on MTHFD2 in these lines. Remarkably, CLIM identified that MTHFD2 was always in the top-ranking collateral lethal targets in all four clusters, as evident from the high therapeutic window index (TWI) and Pareto area under the curve (PAUC) in UQCR11-null cells (FIG. 6f). Thus, despite deviations in the metabolic models of UQCR11-null ovarian cancers that have distinct genetic backgrounds, the importance of MTHFD2 as collateral lethal target was maintained.

Although a varied genetic background can intrinsically influence ovarian cancer metabolism, so too can stromal-rich microenvironments⁵⁰. The collateral lethal targeting of MTHFD2 will be ineffective if the stroma compensates for metabolic deficiencies in the cancer. We first analyzed single-cell RNA sequencing (scRNA-seq) data from ascitic fluid of patients with ovarian cancer and observed that the expressions of MTHFD2 and UQCR11 were strongly negatively correlated across all cell types in ovarian tumours (FIG. 6g). Epithelial (malignant) cells were at the end of the spectrum, with the lowest UQCR11 and highest MTHFD2 expression (FIG. 6g). The pathway-level expression of one-carbon metabolism, including MTHFD2 and encompassing pathways, would reveal their importance in stromal cells when UQCR11 is lost. We first derived ten curated gene sets that would represent pathways related to one-carbon metabolism and ETC from bulk RNA-seq data from the TCGA ovarian tumour cohort. Remarkably, only three of these gene sets were differentially expressed in 19p13.3-deleted ovarian tumours compared with those without deletion from the TCGA cohort. Notably, gene signatures representing the one-carbon pathway including MTHFD2 and serine-glycine-threonine metabolism were both upregulated in 19p13.3-deleted tumours. In contrast, the gene signature reflecting NAD+ biosynthesis was downregulated in 19p13.3-deleted tumours. This corroborated the predictions from CLIM, highlighting the upregulation of MTHFD2 and related pathways in UQCR11-null tumours. Importantly, at the single-cell level the pathway expression of one-carbon and serine-glycine-threonine metabolism gene signatures was higher in epithelial cell types compared with fibroblasts, lymphocytes and macrophages (FIG. 6g). This highlighted that cancer cells rely on cell-intrinsic MTHFD2 and related pathways to restore NAD+ rather than on one-carbon metabolism of stromal cells, especially when they display UQCR11 loss.

These observations were corroborated from bulk RNA-seq data after deconvoluting malignant and stromal populations using CIBERSORTx. Tumours from the TCGA ovarian cancer dataset displayed the presence of five major populations after deconvolution (FIG. 6h). A dimensionally reduced projection of stromal scores for the five populations was obtained using PHATE to visualize the dominance of different stromal types across the TCGA cohort (FIG. 6h). The first PHATE component distinguished tumours based on dominance of epithelial cells (negative PHATE 1 embed-ding) or fibroblasts (positive PHATE 1 embedding), while the the second component dissected the dominance of T cells (negative PHATE 2 embedding) and B cells (positive PHATE 2 embedding). Thus, dividing the PHATE plane into four quadrants allowed us to compare four subgroups of tumour with similar numbers of samples across the groups, each with a distinct stromal population (FIG. 6i). The dominant stromal population of each quadrant was discernible by their respective cell-type-specific score distribution (FIG. 6h, joyplots). Notably, the occurrence of 19p13.3 deletion was indistinguishable across both PHATE dimensions (FIG. 6i), indicating that chromosomal alteration was not associated with stromal composition. Remarkably, pathway expressions of the one-carbon and serine-glycine-threonine metabolism signatures had no discernible differences across the quadrants (FIG. 6j). Further, expression of MTHFD2 was similar across all four quadrants (FIG. 6k). These data indicated that the prevalence of UQCR11-null tumours, and the transcriptional activity of the col-lateral lethal one-carbon metabolism pathway and MTHFD2, were invariant across ovarian tumours with distinct stromal-dominant populations. Notably, expression of UQCR11 in quadrant Q2 was skewed towards the lower end, in line with the dominance of epithelial cells (FIG. 6h,k). These observations were corroborated by microdissection of an ovarian tumour, where malignant tumour segments showing lower UQCR11 expression compared with paired stromal segments also displayed significantly higher one-carbon metabolism pathway expression. In contrast, tumours where UQCR11 expression was similar across malignant and stromal tumour segments displayed no difference in one-carbon metabolism pathway expression. Cumulatively, these observations suggest that MTHFD2 is of importance as a collateral lethal metabolic target in UQCR11-null ovarian cancers, regardless of differences in their intrinsic genetic make-up and extrinsic microenvironments.

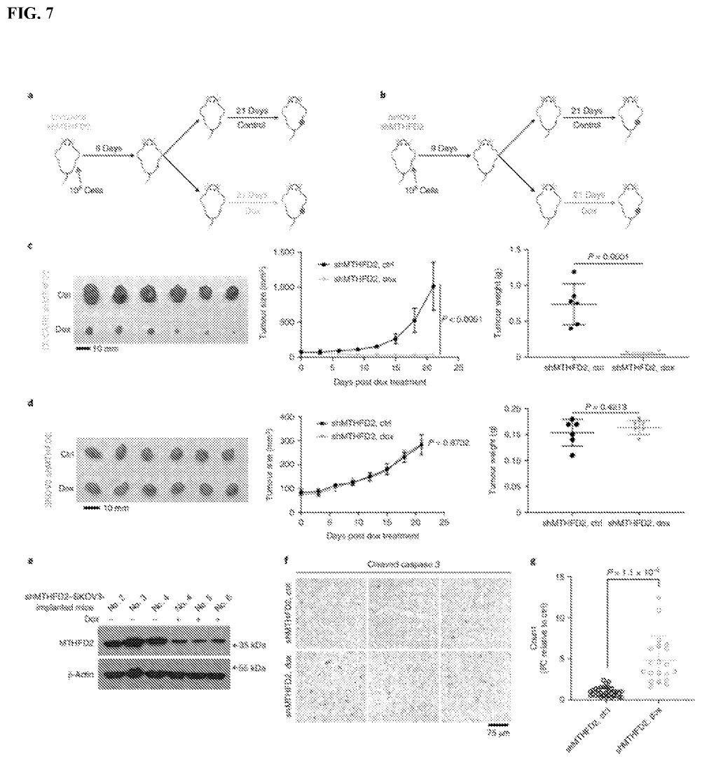

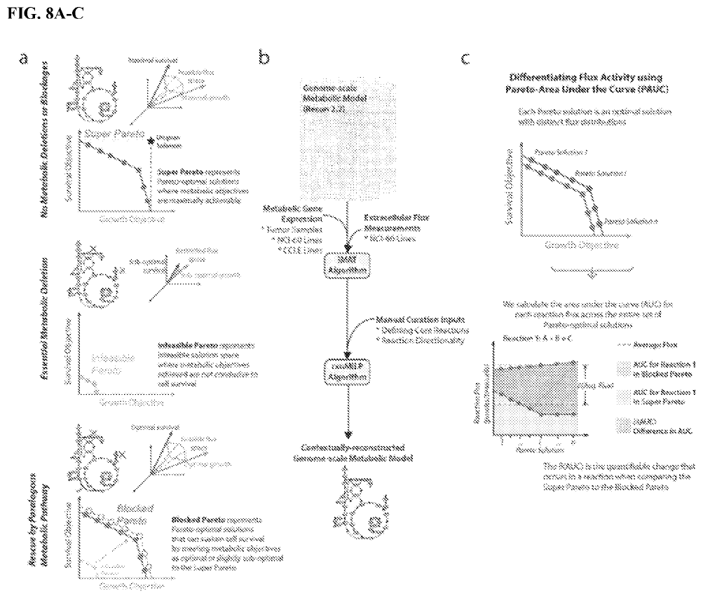

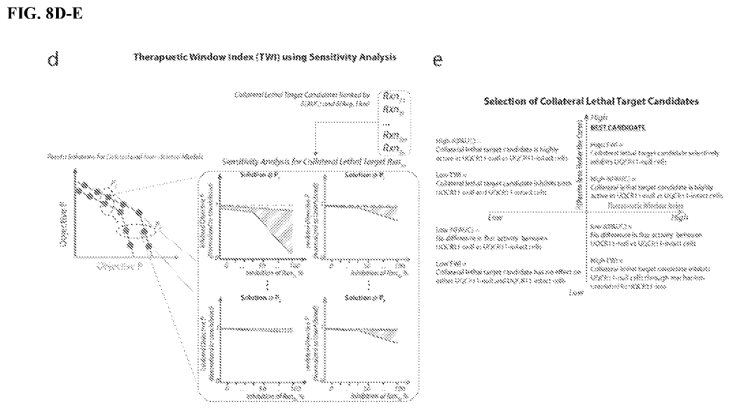

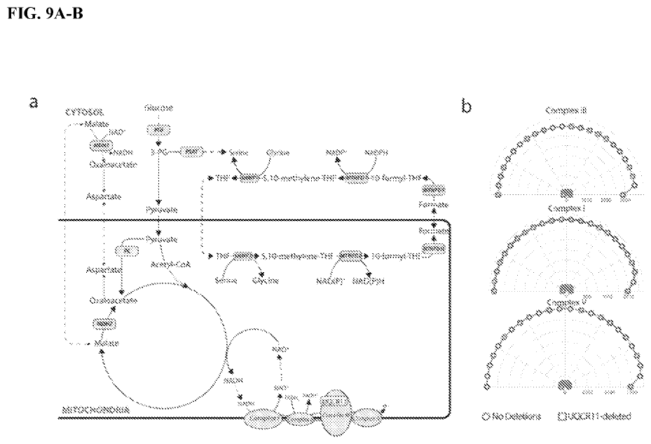

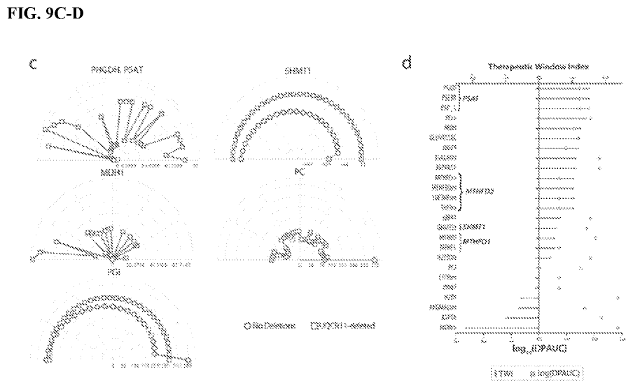

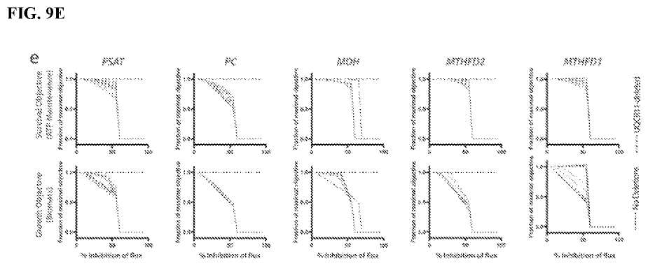

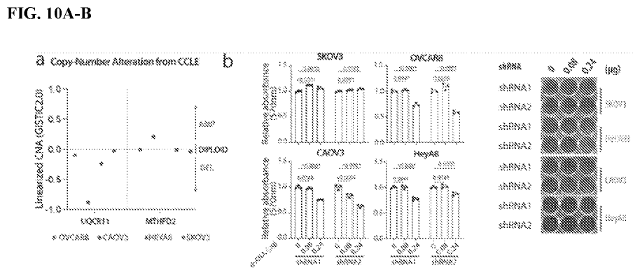

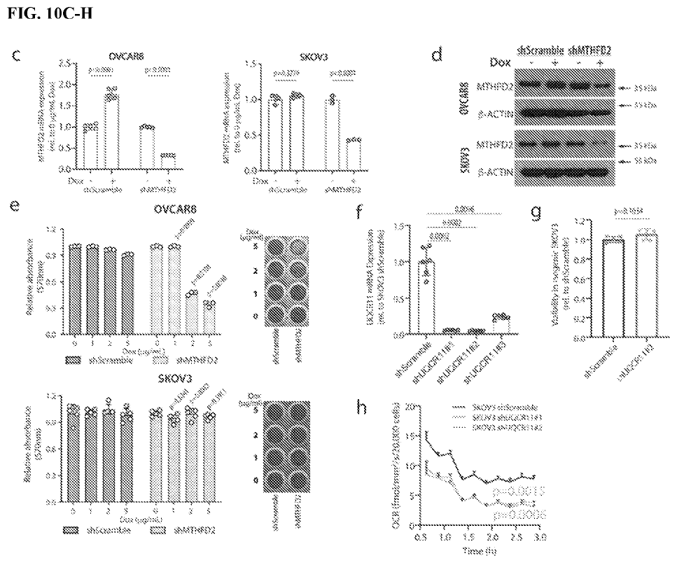

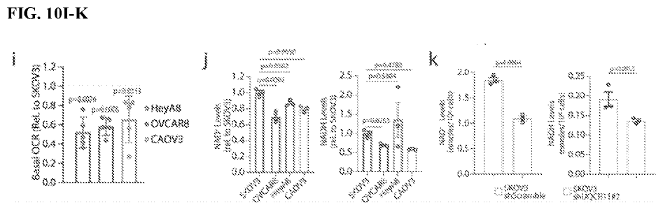

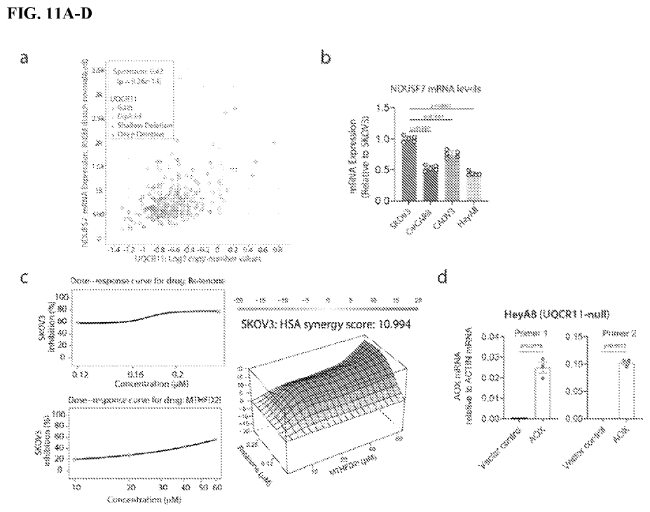

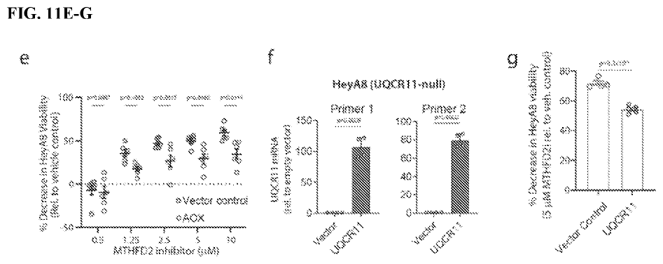

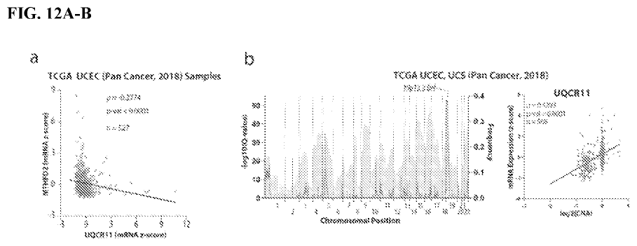

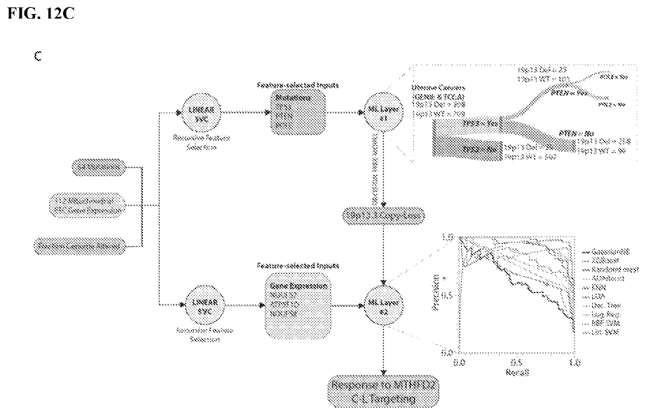

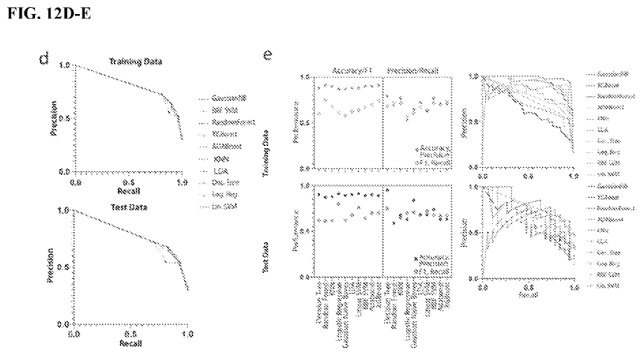

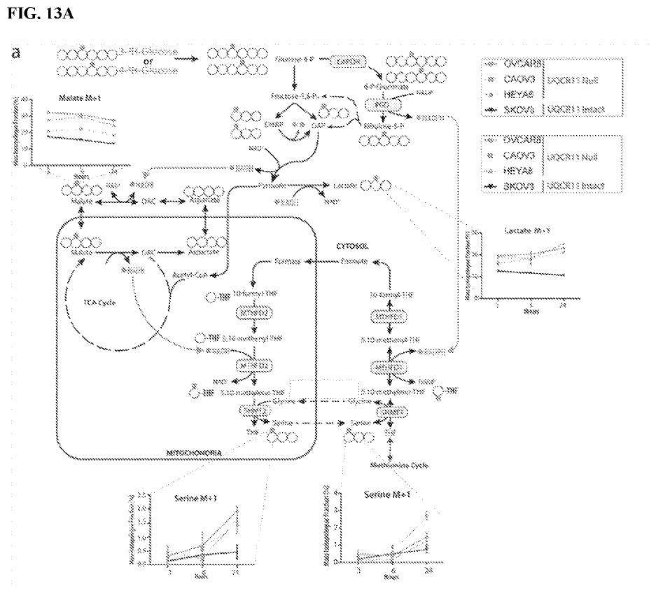

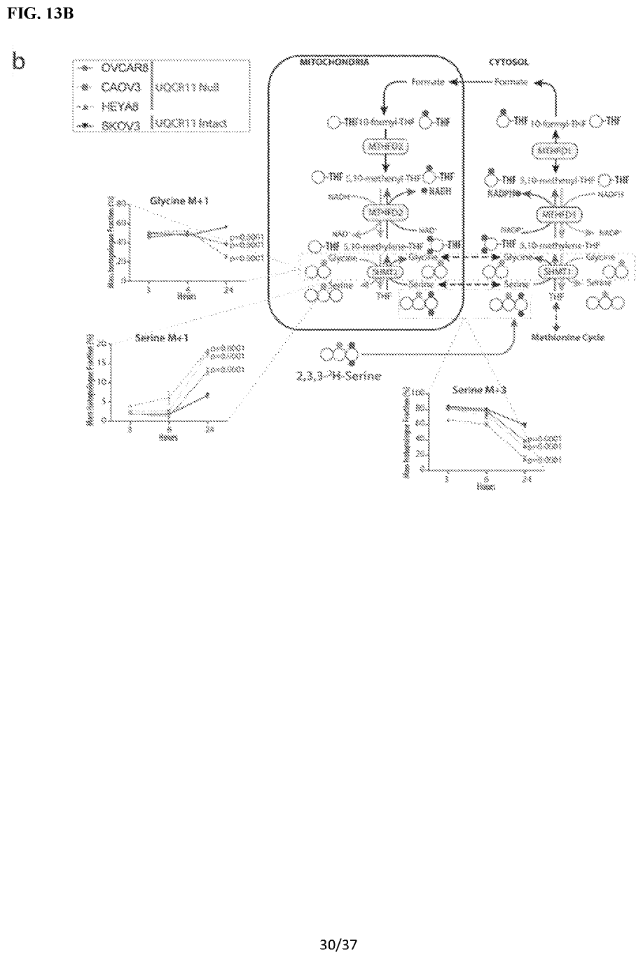

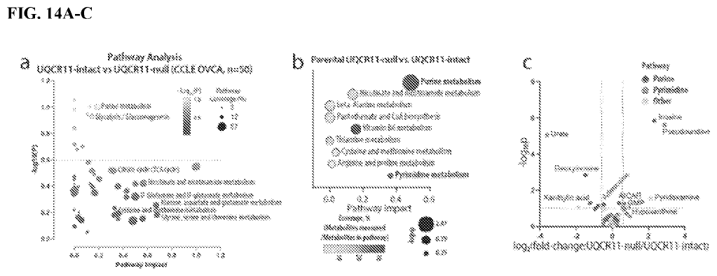

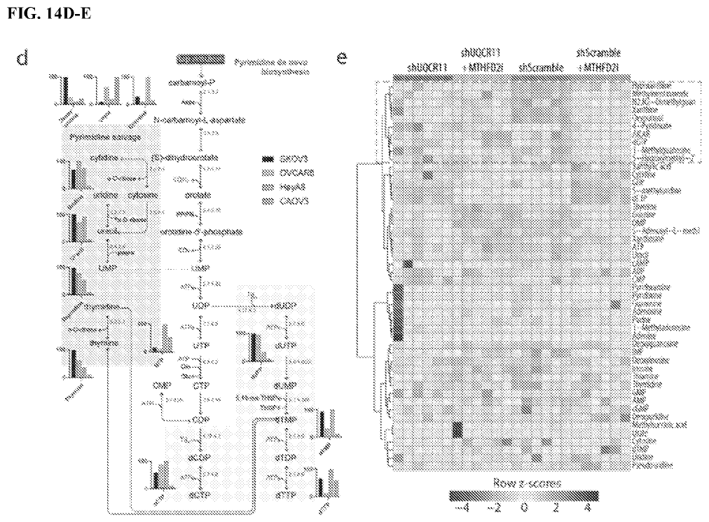

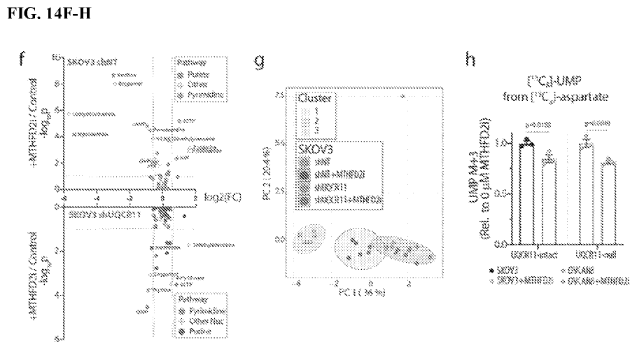

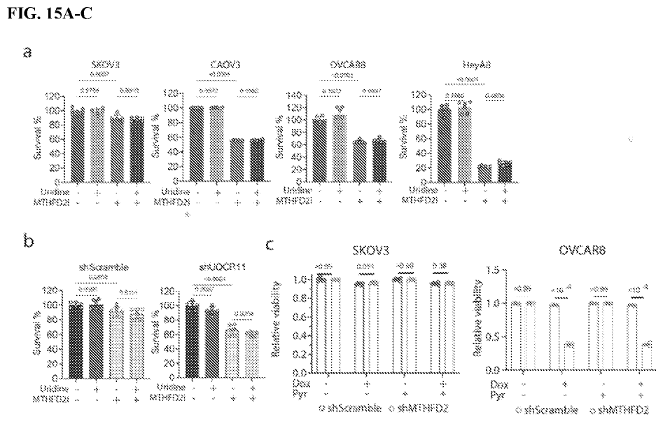

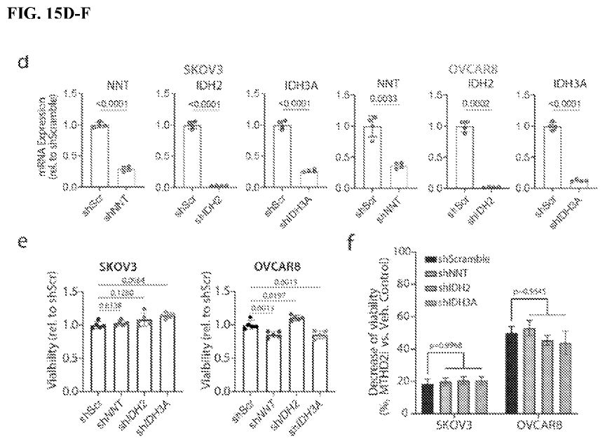

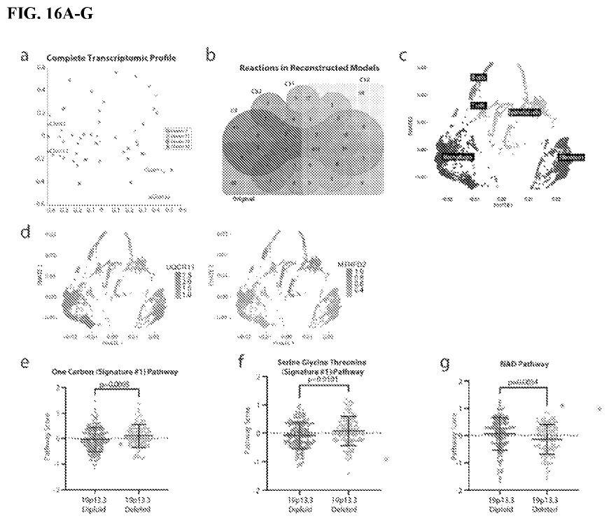

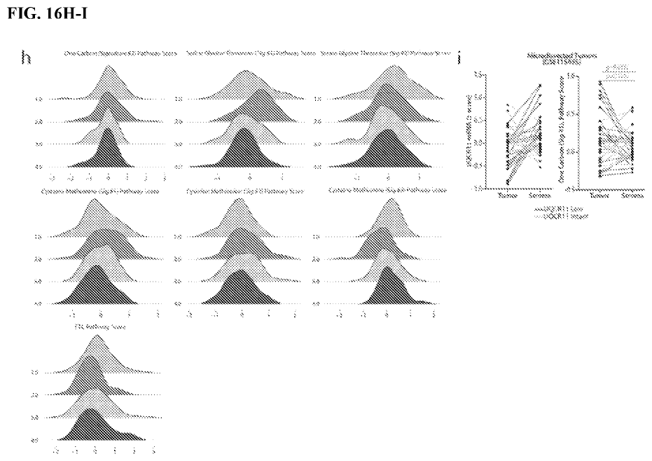

To strengthen the validity of collateral lethal targets predicted by CLIM through in silico and in vitro modalities, we developed subcutaneously implanted tumour models representing UQCR11-null and -intact ovarian tumours with dox-inducible knockdown of MTHFD2 (FIG. 7a,b). As per CLIM's prediction of MTHFD2 essentiality in UQCR11-null cancers, dox-induced knockdown of MTHFD2 dramatically decreased xenograft tumour growth in UQCR11-null but not in UQCR11-intact cells (FIG. 7c,d), despite marked reduction in MTHFD2 expression in UQCR11-intact tumours (FIG. 7e). In line with tumour growth, apoptotic cell populations significantly increased in tissue sections from UQCR11-null tumours (FIG. 7f,g). Collectively, these data reveal the utility of our systems-biology-based workflow to identify collateral lethal metabolic targets (e.g., in ovarian cancer) that show a marked advantage in eliminating tumour growth in vivo.

The utilization of precision medicine-based approaches to discover metabolic targets in cancers has been severely lacking, with families of drugs targeting a given mutation showing drastically differing responses across patients. We therefore used a metabolic systems biology approach to integrate genomic and transcriptomic data with machine learning, genome-scale metabolic flux analysis and state-of-the-art tracing studies to mechanistically identify collateral lethal metabolic vulnerabilities in cancers with specific genomic deletions. The discovery of precision metabolic targets hinges on uncovering redundant metabolic pathways that confer adaptability and survival advantage in cancer cells⁵¹⁻⁵³. Importantly, CLIM predicted collateral lethal metabolic pathways and mechanistically explained the compensation between predicted paralogous pathways. Herein we demonstrate the emergence of MTHFD2 as a collateral lethal target in hypomorphic ovarian cancers with UQCR11 copy loss. MTHFD2 has been considered a potential metabolic target in some cancers due to its overexpression in cancer cells compared with healthy adult cells^{28,54-55}, coupling to folate metabolism-mediated functions³⁹, redox balance^{56,57} and nucleotide metabolism^{28,58}. Despite a broad empirical knowledge base, the successful use of MTHFD2 inhibitors in the clinic has seen limited progress in ovarian cancers.

The finding described herein provide a clinical path for MTHFD2 inhibitors in the context of tumour suppressor gene deletions, especially in cancers with UQCR11 loss. CLIM uncovered an unprecedented non-canonical compensatory function of the MTHFD2 pathway via the oxidative mitochondrial NAD+ balance that is selectively targetable in UQCR11-null ovarian and endometrial cancers.

The modularity of CLIM allows the discovery of mechanistically linked collateral lethal metabolic gene pairs that arise from other chromosomal alterations such as amplifications. Ultimately, CLIM is an end-to-end precision medicine platform that mines clinically relevant chromosomal alterations leading to metabolic vulnerability and predicts collateral lethal metabolic targets linked through mutual metabolic function, thus providing a basis for both empirical and preclinical validation.

### EXAMPLES

Analysis of Clinical and Molecular Data for Tumour Samples. Data Access.

Molecular and clinical data were obtained via CBioPortal61,62 for TCGA Pan-Cance Atlas samples, CCLE (Broad 2019), NCI-60 Cell Lines and AACR Project GENIE63 (cohort v.7.0-Data processing. For all bioinformatics and machine learning analyses, only primary tumour samples were retained. Gene expression data were downloaded as z-scores relative to all samples for TCGA datasets. Segmentation files were used as input for the GISTIC2.0 algorithm14 deployed on GenePattern Cloud64 with default settings, to obtain linearized copy number alteration values and regions of statistically significant focal deletions.

Metabolic genes. A reference list of metabolic genes for all analyses was obtained from the Kyoto Encyclopedia of Genes and Genomes (KEGG) database. The dataset of metabolic pathways and gene sets were manually curated to remove pathways involved in xenobiotic metabolism, proteasomal processing and signalling.

Geneticparalogues. Paralogous genes for the set of deleted metabolic genes were obtained from the Ensembl database (https://uswest.ensembl.org/Help/View?id=137), determined by the Gene Ontology/Paralogy prediction method. Only those metabolic genes with at least one predicted paralogue in the database were counted as genes with paralogues.

Circosplots. Circos plots were generated using circos-0.69-9 software65. Average copy number gain and loss were used to populate the outermost rings; the middle ring was populated with the GISTIC2.0-log 10(residual q-value) to represent focal deletions of statistical significance; the innermost rings were populated by the number of genes and metabolic genes lying within the peaks of focal deletion detected by GISTIC2.0. The labels identify deleted metabolic genes with their official Human Gene Nomenclature Committee symbol.

Correlation. Metabolic genes deemed to be lost due to chromosomal deletions were selected based on their frequency of loss, as determined by GISTIC2.0 and whether copy loss translated to loss of gene expression. This condition is a representation of loss of metabolic function and is estimated by Spearman's rank correlation between the linearized copy number alteration and z-score of mRNA expression.

Survival analysis. Cohorts were divided according to CNA or gene expression, as described. Plots were generated and log-rank statistical tests were performed using the function MatSurv (https://github.com/aebergl/MatSurv)⁶⁶.

Venn diagrams. Diagrams for pictorial representation of overlaps across the reconstructed ovarian cancer metabolic models were generated using the open-source Venn diagram web-tool http://bioinformatics.psb.ugent.be/webtools/Venn/.

Cancer dependency map scores. Genetic dependency data (DEMETER2 scores) for MTHD2 in cell lines were downloaded from the DepMap portal (CRISPR Public 20Q1).

RSF analysis in TCGA ovarian tumours. Ovarian tumour samples from the TCGA Pan-Cancer dataset with both gene expression and clinical data available were analysed (n=295). RSF models use the machine learning concept of forest ensembles learners for application to right-censored survival data. Here we applied RSF to discover risk factors—in particular, metabolic genes that predict the prognosis of patients with 19p13.3-deleted ovarian tumours. RSF was implemented in R (CRAN 4.0.3) using the package randomForestSRC (cran.r-project.org/web/packages/randomForestSRC). In addition to patient age and expression of enzyme-encoding genes, the following KEGG pathways were used as risk factors for input to the RSF model: one-carbon pool by folate (hsa00670), cysteine and methionine metabolism (has 00270), serine, glycine and threonine metabolism (has00260), ECR reaction (hsa00190), TCA cycle (hsa00020) and NAD pathway (hsa00760). Variable importance and mortality (overall survival) prediction plots were generated using the function vimp. Heatmap and variable importance plots are shown for importance values >0.001.

NNMF for classification of molecular features and patient strata. We used a recursive NNMF approach on integrated mutation and copy number data from 629 patients with ovarian cancer to cluster those with distinct molecular traits-specifically, deep deletions with metabolic genes. Unsupervised NNMF was performed on a molecular feature-by-sample matrix using a method adapted from Moffitt et al.⁶⁷ and scripted in MATLAB (Mathworks, 2018a). Molecular features were defined by copy number alteration values of focal loci and frequently occurring mutations. Mutation data were represented as binary, where 0 represents no mutation and a positive ‘score’ represents all but non-sense mutations. The score was set to the 0.5th percentile of linearized copy number values, to ensure that mutations would be ranked as importantly as deletions by the unsupervised algorithm. To ensure a non-negative matrix and to prioritize focal deletions, the combined data were transformed as 2(−x), where x represents every matrix element. For the ovarian cancer dataset, the top 20 frequently occurring mutations as reported by the database COSMIC v.91 (ref. 68) (cancer. sanger.ac.uk) were retained in the feature matrix.

UpSetplots. UpSet plots were used to visualize overlap of molecular features from the feature loading in four clusters where 19p13.3 deletion was identified as a defining feature identified by NMF-based clustering of ovarian tumour samples from TCGA. The plots were generated with the UpSetR⁶⁹ package implemented in R (CRAN 3.6.3).

Multi-objective flux analysis to identify collateral lethal metabolic targets. Multi-objective metabolic flux analysis. We then employed multi-objective flux balance analysis to compute metabolic activity that represents the adaptable nature of cancer cell metabolism. We used Pareto optimization to compute metabolic fluxes under competing metabolic objectives—for example, cellular growth (that is, biomass synthesis) and cell survival (that is, ATP and redox maintenance)^{70,71}.

These metabolic objectives are critical in tumorigenesis due to high bioenergetic demands in cancer cells competing for the limited nutrients available in the tumour microenvironment. The design of the multi-objective metabolic flux analysis has been developed in our laboratory and demonstrated in a hepatocyte system^{70,71}. The flux balance analysis algorithm implemented in the toolbox COBRA (v.3.0)72 was modified to a multi-objective optimization problem. We employ the normalized normal constraint method derived for engineering problems⁷³, because it is scale independent in the objective function space. This is especially important when the system variables are intracellular fluxes, which can range across multiple orders of magnitude.

Normalized constraint method for generation of Pareto-optimal solutions (two cellular objectives). To obtain the Pareto frontier for estimation of optimal flux distributions at the two metabolic objectives, known as the anchor points, maximal biomass synthesis is represented by biomass reaction flux and maximal ATP by maintenance flux, DM_ATP_c flux. The two distinct optimization problems are defined in equation (1):

$$maximize g 1 or g 2 ( 1 )$$

$$subject to :$$

$$Sv = 0$$

$${v}_{lb} \leq v \leq {v}_{ub}$$

The anchor points, or the optimal solutions of the metabolic objectives, are represented by g*₁ and g*₂, and their corresponding optimal intracellular flux distributions are v₁* and v₂*. Normalization is done with respect to the ranges of objective function values and is represented as ǧ. The utopia point, which is the theoretical optimal solution for both objectives is g^{u}(equation (2)) and the normalization factors are estimates as in equation (3):

$${g}^{u} = {[ \begin{matrix}{g}_{1}^{*} & {g}_{2}^{*}\end{matrix} ]}^{T} \qquad ( 2 )$$

$${l}_{1} = {g}_{1} ( {v}_{2}^{*} ) - {g}_{1}^{*} \qquad ( 3 a )$$

$${l}_{2} = {g}_{2} ( {v}_{1}^{*} ) - {g}_{2}^{*} \qquad ( 3 b )$$

The optimization problem and objective function space (equation (4)) used to generate the Pareto frontier are transformed such that the objective function is normalized in terms of g^{u}, l₁ and l₂:

$${\overline{g}}^{T} = [ \frac{{g}_{1} ( v ) - {g}_{1}^{*}}{{l}_{1}} \frac{{g}_{2} ( v ) - {g}_{2}^{*}}{{l}_{2}} ] \qquad ( 4 )$$

The reduction constraint (equation (5)) is derived using orthogonality with the utopia line, N₁ that is the vector in the direction of

$$\overline{{g}_{1}^{*}} and \overline{{g}_{2}^{*}} : \qquad ( 5 )$$

$$\overline{{N}_{1}} = \overline{{g}_{2}^{*}} - \overline{{g}_{1}^{*}}$$

The utopia line is divided into m₁−1 segments and the reduction constraint vector is divided into equal segments with ∂=1/(m₁−1) normalized increments to obtain the utopia line defined as the j^{th} segment of the vector connecting the anchor points gₖ*, corresponding to each Pareto solution, Pⱼ. Each segment is obtained from the weighted fraction, αₖⱼ, of the optimal solutions gₖ*, where j∈[1, m₁]∩Z (equations (6-7)):

$$\overline{{X}_{Pj}} = {\sum }_{k = 1}^{2} {α}_{kj} \overline{{g}_{k}^{*}} \qquad ( 6 a )$$

$$0 \leq {α}_{kj} \leq 1 and {\sum }_{k = 1}^{2} {α}_{kj} = 1 \qquad ( 6 b )$$

The normal line N₁ is moved across X_{Pj} by incrementing αₖⱼ (equation (7)) to restrict the feasible region and leading to a new feasible minimum point:

$${α}_{kj} = {\{ i * \partial \}}_{i = 0}^{m - 1} \qquad ( 7 )$$

Solving the succession of optimization problems (equation (8)) with the additional normal constraint (equation (9)) will generate a set of Pareto points for corresponding values of each incremental change in feasible regions:

$$minimize \{ \overline{{g}_{2} ( v )} \} \qquad ( 8 )$$

$$subject to :$$

$${N}_{1} \cdot {( \overline{g ( v )} - \overline{{X}_{Pj}} )}^{T} \leq 0 \qquad ( 9 )$$

$$Sv = 0$$

$${v}_{lb} \leq v \leq {v}_{ub}$$

The Pareto points obtained are in normalized space and need to be transformed to original coordinates using the inverse mapping relation:

$${g}^{Pareto} = {[ \begin{matrix}( \overline{{g}_{1}} {l}_{1} + {g}_{1}^{*} ) & ( \overline{{g}_{2}} {l}_{2} + {g}_{2}^{*} )\end{matrix} ]}^{T}$$

The set of Pareto-optimal solutions, g^{Pareto}, are such that increases in one objective function come at the cost of the other. First, Pareto-optimal solutions for the condition with no deletions are simulated using the cancer-specific metabolic model from the previous step.

Metabolic gene deletion is emulated by first limiting all reactions in the model encoded by the deleted gene to 10% of the flux value in the non-deleted condition. To ensure that all reactions are limited, we use the function findRxnsFromMets in COBRA and the gene-protein reaction (GPR) to find reactions that are catalysed by (i) only the gene-encoded enzyme or (ii) a complex that requires the subunit encoded by the deleted gene, represented in the GPR rules as ‘and’.

PAUC. The Pareto area-under-the-curve (PAUC) is a quantification of the extent to which each flux changes across the entire Pareto frontier. The difference between the PAUC values of fluxes is derived by comparing two conditions, in this case Pareto-optimal fluxes in UQCR11-intact cells and UQCR11-null ovarian cancer models. PAUC^{C}ₖ is calculated for each condition across all Pareto-optimal solutions, as described in equations (10) and (11):

$${PAUC}_{k}^{C} = \frac{1}{{N}_{P}^{C}} {\sum }_{i = 1}^{{N}_{P}^{C} - 1} \frac{{v}_{k , i + 1}^{C} + {v}_{k , i}^{C}}{2} , k ∈ intracellular fluxes , C ∈ \{ deleted , intact \} \qquad ( 10 )$$

$$Δ {PAUC}_{k} = {PAUC}_{k}^{DEL} - {PAUC}_{k}^{INT} \qquad ( 11 )$$

TWI (sensitivity analysis). The TWI is an estimation of the extent to which a collateral lethal candidate target (v_{CL}), when inhibited, would affect cells with corresponding metabolic deletion compared with cells without deletion. First, the sensitivity of perturbing (inhibiting) v_{CL} is estimated on each randomly chosen Pareto solution, p, as the change in the objective function value for every incremental inhibition of v_{CL} and δv_{CL} (equation (12)). All fluxes except v_{CL} are restricted to within 25% of their original values. The average sensitivity to v_{CL} is calculated as the square-root of the 2-norm of sensitivities for each objective function (equation (13)). The TWI is the difference in average sensitivities to perturbation of v_{CL} between deleted and non-deleted Pareto-optimal solutions (equation (14)):

$$\frac{Δ {f}_{P}^{C}}{Δ {v}_{CL}} = {\sum }_{i = 0}^{I} \frac{{f}_{P}^{C} ( {v}_{CL} - ( i + 1 ) * δ {v}_{CL} ) - {f}_{P}^{C} ( {v}_{CL} - ( i ) * δ {v}_{CL} )}{\partial Δ {v}_{CL}} , \qquad ( 12 )$$

$$I = floor ( 0.9 * \frac{{γ}_{CL}}{δ {v}_{CL}} )$$

$${s}_{CL}^{C} = \sqrt[i]{{\sum }_{p} \frac{Δ {f}_{P}^{1 , C}}{Δ {v}_{CL}} + {\sum }_{P} \frac{Δ {f}_{P}^{2 , C}}{Δ {v}_{CL}}} \qquad ( 13 )$$

$$TWI = {s}_{CL}^{DEL} - {s}_{CL}^{INT} \qquad ( 14 )$$

p∈{randomly chosen Pareto solutions}
   C∈{deleted, intact}

Cell culture. CAOV3 were sourced from the American Type Culture Collection. SKOV3, OVCAR8 and HeyA8 were obtained from the MDACC Cell-line Core Facility and cultured in high-glucose DMEM (Corning, no. 10-017-CM) supplemented with 10% fetal bovine serum (Corning, no. 35-010-CV) and penicillin/streptomycin (Hyclone, no. SV30010) at 37° C. in a 5% CO2 incubator. All cell lines were confirmed as having no mycoplasmal contamination using a Mycoplasma Detection kit (Lonza, no. LT07-118).

Tumour implantation and treatment. Eight-week-old female NU/J mice were purchased from Jackson Laboratories and housed under pathogen-free conditions; by subcutaneous flank injection, 1×106 dox-inducible shMTHFD2 OVCAR8 or SKOV3 cells in PBS were introduced. Six to nine days after injection of tumour cells, mice were randomly divided into two groups followed by regular food or dox+ food. Tumour size was measured every 3 days using a caliper, and tumour volume was calculated using the standard formula 0.5×L×W2, where L is the longest diameter and W is the shortest diameter. Based on the animal protocol approved by the Institutional Animal Care and Use Committee of Indiana University School of Medicine, any mouse with tumour size ≥1,500 mm3 was euthanized. In this study, all tumours grown in the mouse model at the endpoint were <1,500 mm3. Housing conditions: ambient room temperature 22±2° C., 12/12 h light/dark cycle (lights on at 07:00) and 30-70% relative humidity.

Multilayer machine learning model to predict 19p13.3 loss and utility of collateral lethal targeting of MTHFD2 in endometrial cancer. We used a two-model system to predict the expected MTHFD2 response from tumour mutation data and expression data for ETC genes. All model training and evaluation was performed via the Python toolbox scikit-learn (scikit-learn.org/) and the XGBoost library⁷⁴. To train the first model that predicts 19p13.3 loss from gene mutation, we used a list of 54 mutations commonly found in UCEC as reported by COSMIC v.91 (ref. 68). We combined mutation data from 1,176 samples from the GENIE database⁶³ and 579 from the UCEC TCGA Pan-Cancer database. We divided the data from the TCGA cohort into a 20% test set for overall model testing and an 80% set for training and testing of the first model. We combined 80% of TCGA mutation data with those from GENIE and split the combined data into an 80% training dataset and a 20% test dataset. We used the chi-squared test and fourfold recursive feature elimination (RFE) to perform feature selection. The chi-squared test was implemented via chi2( ) and RFE was implemented via RFECV, with the estimator set to scv (linear support vector classifier) and scoring set to accuracy. Training data for three selected mutations (PTEN, TP53 and POLE) were used to train different classification models, with fivefold cross-validation for hyperparameter selection (classification models: KNN, linear SVM, rbf SVM, AdaBoost, XGBoost, decision tree, random forest, logistic regression, naive bayes, LDA). We applied the models to the test set and selected a decision-tree model that had the best performance on the testing and training sets. Next, we used z-scored expression data for 108 ETC genes for the 579 tumour samples and the 19p13.3 prediction from the first model to train the second model. We used the 20% dataset previously set aside for testing and the remainder for training.

OCR measurement. Live-cell OCR via Resipher. A total of 20,000 cells were seeded into flat-bottomed, 96-well treated plates (Falcon, no. 353072) in complete DMEM culture medium and incubated for 5 h to facilitate cell attachment. The medium was then replaced with another containing different rotenone concentrations (31.0, 62.5, 125, 250 and 500 nM) for 1 h, after which medium was replaced by fresh containing rotenone and the MTHFD2 inhibitor, DS18561882 (0 or 60 μM), and the Resipher oxygen-sensing lid (Lucid Scientific) was attached. Live OCR was measured for up to 16 h after attaching the lid.

Mito stress test with the Seahorse analyser. Mitochondrial OCR was measured using a Seahorse XFe96 analyser (Agilent Technologies). Cells were seeded in 96-well Seahorse cell culture plates and incubated at 37° C. with 5% CO2 overnight. Cells were exposed to different concentrations of rotenone (31.0, 62.5, 125, 250 and 500 nM) for 12 h, followed by DS18561882 (MedChemExpress, no. HY-130251) for 3 h. The assay was performed according to the manufacturer's protocol. In brief, medium was replaced with 180 μl of Seahorse XF medium free from serum and sodium bicarbonate. The Seahorse plate was then incubated in a CO2-free incubator at 37° C. for 45 min before being placed in a Seahorse XFe96 analyser. Sequential measurements of OCR were performed by the injection of the following inhibitors-oligomycin, FCCP and rotenone plus antimycin A through ports A, B and C, respectively, to calculate basal OCR under different stresses. All data were normalized to total cell protein, as measured by bicinchoninic acid assay.

Flux analysis via [2H]-labelled substrates and metabolomics in vitro. Polar metabolite extraction for the analysis of amino acids, NAD+ and NADH was performed as in our previous studies^{50,75-79}. Cells were cultured in six-well plates with either 3-[2H]-glucose (Cambridge Isotope Labs, no. DLM-9294-PK), 4-[2H]-glucose (Cambridge Isotope Labs, no. DLM-9294-PK) or 2,3,3-[2H]-glucose (Cambridge Isotope Labs, no. DLM-9294-PK) and then, after 3, 6 or 24 h, quenched with 800 μl of ice-cold methanol/water (1:1) solution containing 1 μg of norvaline. Cells were scraped while on ice, followed by the addition of 800 μl of high-performance liquid chromatography-grade chloroform to remove lipid content from the sample matrix. Finally, cell extracts were transferred to microcentrifuge tubes and vortexed vigorously for 30 min at 4° C. The extracted samples were dried in a SpeedVac (ThermoScientific) for 2-4 h without heat and stored at −80° C. The drying process was continuously monitored and stopped as soon as samples were completely dry, to minimize metabolite degradation.

scRNA-seq data clustering for gene and pathway score projections. Data access. scRNA-seq data were obtained from an online repository with accession no. GSE146026 (ref 80). The dataset consists of 11,000 single cells from 22 ovarian cancer ascites samples.

Dataprocessing. Data normalization and visualization were performed using the packages Rmagic⁸¹ and PhateR⁸² implemented in R (CRAN 4.0.4). Raw counts are quantified as log 2(TPM/IO+1), where TPM denotes transcripts per million. We performed an initial quality control that involves removal of genes expressed in fewer than ten cells and exclusion of cells with <1,000 counts. Li normalization was performed on quality-control-treated data, followed by square-root transformation of the resulting matrix. This was followed by final normalization using the ‘magic’ function of the package Rmagic⁸¹. Briefly, the magic function will first right-multiply the input matrix with a coefficient matrix called Markov affinity matrix and then rescale the matrix. Finally, the various clusters present in the datasets were visualized using the function phate in the package PhateR⁸² with the following parameters: knn=5, decay=100 and t=20. Clusters were identified based on the following gene markers: epithelial (KRT17, KRT4, EPCAM, MMP7); fibroblasts (PDPN, DCN, THY1, SNAI2); macrophages (CD14, AIF1, CSF1R, CD163); T cells (CD2, GZMB); and B cells (CD19, CD79A). Plots were generated with ggplot2 (ref. 83) implemented in R (CRAN 4.0.4).

Estimation of cell type proportions in ovarian bulk RNA-seq data using CIBERSORTx. Bulk RNA-seq data for ovarian serous cystadenocarcinoma from the TCGA Pan-Cancer Atlas were obtained via CBioportal^{62,84}. scRNA-seq data were obtained from an online repository with accession no. GSE118828 (ref. 85). scRNA-seq was obtained from 14 samples collected from nine patients with varying grades of ovarian cancer. Raw ovarian scRNA-seq data were obtained as unique molecular identifier (UMI) gene counts per cell. Using the pipeline Seurat v.4 (ref. 86) in R (CRAN 4.04), outlier cells with total UMI count <200 or >5,000 were deselected. In addition, dying cells with >5% mitochondrial genes were excluded from further analysis. Quality-control-treated data were log-normalized with a scale factor of 10,000, and the top 2,000 variable features (genes) among cells were identified. Principal component analysis (PCA) was performed on variable features, with the first 20 PCA used for clustering with the shared nearest-neighbour algorithm at a resolution of 0.5 followed by visualization using the uniform manifold approximation and projection dimensional reduction algorithm. The generated single-cell reference matrix file and bulk RNA-seq were the two main inputs to the online CIBERSORTx digital cytometry⁸⁷. Cell fractions were computed in absolute mode with 500 permutations.

### REFERENCES

1. Pertesi, M. et al. Essential genes shape cancer genomes through linear limitation of homozygous deletions. Commun. Biol. 2, 262 (2019).
2. Dey, P. et al. Genomic deletion of malic enzyme 2 confers collateral lethality in pancreatic cancer. Nature 542, 119-123 (2017).
3. Muller, F. L., Aquilanti, E. A. & DePinho, R. A. Collateral lethality: a new therapeutic strategy in oncology. Trends Cancer 1, 161-173 (2015).
4. Muller, F. L. et al. Passenger deletions generate therapeutic vulnerabilities in cancer. Nature 488, 337-342 (2012).
5. Sanderson, S. M., Mikhael, P. G., Ramesh, V., Dai, Z. & Locasale, J. W. Nutrient availability shapes methionine metabolism in p16/MTAP-deleted cells. Sci. Adv. 5, eaav7769 (2019).
6. Ryland, G. L. et al. Loss of heterozygosity: what is it good for?BMC Med. Genet. 8, 45 (2015).
7. Liu, Y. et al. TP53 loss creates therapeutic vulnerability in colorectal cancer. Nature 520, 697-701 (2015).
8. Hart, T., Koh, C. & Mofat, J. Coessentiality and cofunctionality: a network approach to learning genetic vulnerabilities from cancer cell line ftness screens. Preprint at bioRxiv https://doi.org/10.1101/134346 (2017).
9. Wang, T. et al. Gene essentiality profling reveals gene networks and synthetic lethal interactions with oncogenic Ras. Cell 168, 890-903 (2017).
10. Wainberg, M. et al. A genome-wide atlas of co-essential modules assigns function to uncharacterized genes. Nat. Genet. 53, 638-649 (2021).
11. Ku, A. A. et al. Integration of multiple biological contexts reveals principles of synthetic lethality that afect reproducibility. Nat. Commun. 11, 2375 (2020).
12. Bayraktar, E. C. et al. Metabolic coessentiality mapping identifes C12orf49 as a regulator of SREBP processing and cholesterol metabolism. Nat. Metab. 2, 487-498 (2020).
13. Molina, J. R. et al. An inhibitor of oxidative phosphorylation exploits cancer vulnerability. Nat. Med. 24, 1036-1046 (2018).
14. Mermel, C. H. et al. GISTIC2.0 facilitates sensitive and confdent localization of the targets of focal somatic copy-number alteration in human cancers. Genome Biol. 12, R41 (2011).
15. Lee, J. Y. et al. A distinct region of chromosome 19p13.3 associated with the sporadic form of adenoma malignum of the uterine cervix. Cancer Res. 58, 1140-1143 (1998).
16. Wang, Z. J. et al. Allele loss and mutation screen at the Peutz-Jeghers (LKB1) locus (19p13.3) in sporadic ovarian tumours. Br. J. Cancer 80, 70-72 (1999).
17. Chang, K. et al. Te Cancer Genome Atlas Pan-Cancer analysis project. Nat. Genet. 45, 1113-1120 (2013).
18. Fernindez-Vizarra, E. & Zeviani, M. Nuclear gene mutations as the cause of mitochondrial complex III defciency. Front. Genet. 6, 134 (2015).
19. Owens, K. M., Kulawiec, M., Desouki, M. M., Vanniarajan, A. & Singh, K. K. Impaired OXPHOS complex III in breast cancer. PLoS ONE 6, e23846 (2011).
20. Hsu, C.-C., Tseng, L.-M. & Lee, H.-C. Role of mitochondrial dysfunction in cancer progression. Exp. Biol. Med. 241, 1281-1295 (2016).
21. Acin-Perez, R. et al. Respiratory complex III is required to maintain complex I in mammalian mitochondria. Mol. Cell 13, 805-815 (2004).
22. DeBerardinis, R. J. & Chandel, N. S. Fundamentals of cancer metabolism. Sci. Adv. 2, e1600200 (2016).
23. Weinberg, S. E. & Chandel, N. S. Targeting mitochondria metabolism for cancer therapy. Nat. Chem. Biol. 11, 9-15 (2015).
24. Kawai, J. et al. Discovery of a potent, selective, and orally available MTHFD2 inhibitor (DS18561882) with in vivo antitumor activity. J. Med. Chem. 62, 10204-10220 (2019).
25. Martinez-Reyes, I. et al. Mitochondrial ubiquinol oxidation is necessary for tumour growth. Nature 585, 288-292 (2020).
26. Martinez-Reyes, T. et al. TCA cycle and mitochondrial membrane potential are necessary for diverse biological functions. Mol. Cell 61, 199-209 (2016).
27. Sommer, N. et al. Bypassing mitochondrial complex III using alternative oxidase inhibits acute pulmonary oxygen sensing. Sci. Adv. https://doi.org/10.1126/sciadv.aba0694 (2020).
28. Zhu, Z. & Leung, G. K. K. More than a metabolic enzyme: MTHFD2 as a novel target for anticancer therapy?Front. Oncol. https://doi.org/10.3389/fonc.2020.00658 (2020).
29. Liu, M. et al. Te identifcation of key genes and pathways in glioma by bioinformatics analysis. J. Immunol. Res. 2017, U.S. Pat. No. 1,278,081 (2017).
30. Contreras, C. M. et al. Lkbl inactivation is sufcient to drive endometrial cancers that are aggressive yet highly responsive to mTOR inhibitor monotherapy. Dis. Model. Mech. 3, 181-193 (2010).
31. Peiia, C. G. & Castrillón, D. H. In Molecular Genetics of EndometrialCarcinoma (ed. Ellenson, L. H.) 211-241 (Springer International Publishing, 2017).
32. Ghandi, M. et al. Next-generation characterization of the Cancer Cell Line Encyclopedia. Nature 569, 503-508 (2019).
33. Lin, Y. H. et al. An enolase inhibitor for the targeted treatment of ENOl-deleted cancers. Nat. Metab. 2, 1413-1426 (2020).
34. Leonard, P. G. et al. SF2312 is a natural phosphonate inhibitor of enolase. Nat. Chem. Biol. 12, 1053-1058 (2016).
35. Fox, J. T. & Stover, P. J. In Vitamins & Hormones Vol. 79 (ed. Litwack, G.) 1-44 (Academic Press, 2008).
36. Lewis, CarolineA. et al. Tracing compartmentalized NADPH metabolism in the cytosol and mitochondria of mammalian cells. Mol. Cell 55, 253-263 (2014).
37. Gustafsson Sheppard, N. et al. Te folate-coupled enzyme MTHFD2 is a nuclear protein and promotes cell proliferation. Sci. Rep. 5, 15029 (2015).
38. Ducker, G. S. et al. Reversal of cytosolic one-carbon fux compensates for loss of the mitochondrial folate pathway. Cell Metab. 23, 1140-1153 (2016).
39. Morscher, R. J. et al. Mitochondrial translation requires folate-dependent tRNA methylation. Nature 554, 128-132 (2018).
40. Yang, L. et al. Serine catabolism feeds NADH when respiration is impaired. Cell Metab. 31, 809-821 (2020).
41. Fan, J. et al. Quantitative fux analysis reveals folate-dependent NADPH production. Nature 510, 298-302 (2014).
42. Lim, E. W., Parker, S. J. & Metallo, C. M. In Metabolic Flux Analysis in Eukaryotic Cells: Methods and Protocols (ed. Nagrath, D.) 51-71 (Springer US, 2020).
43. Ma, Y., Wang, L. & Jia, R. Te role of mitochondrial dynamics in human cancers. Am. J. Cancer Res. 10, 1278-1293 (2020).
44. Macleod, K. F. Mitophagy and mitochondrial dysfunction in cancer. Annu. Rev. Cancer Biol. 4, 41-60 (2020).
45. Li, H. et al. Te landscape of cancer cell line metabolism. Nat. Med. 25, 850-860 (2019).
46. Birsoy, K. et al. An essential role of the mitochondrial electron transport chain in cell proliferation is to enable aspartate synthesis. Cell 162, 540-551 (2015).
47. Khutornenko Anastasia, A. et al. Pyrimidine biosynthesis links mitochondrial respiration to the p53 pathway. Proc. Natl Acad. Sci. USA 107, 12828-12833 (2010).
48. Spinelli, J. B. et al. Fumarate is a terminal electron acceptor in the mammalian electron transport chain. Science 374, 1227-1237 (2021).
49. Lee, W. D., Mukha, D., Aizenshtein, E. & Shlomi, T. Spatial-fuxomics provides a subcellular-compartmentalized view of reductive glutamine metabolism in cancer cells. Nat. Commun. 10, 1351 (2019).
50. Yang, L. et al. Targeting stromal glutamine synthetase in tumors disrupts tumor microenvironment-regulated cancer cell growth. Cell Metab. 24, 685-700 (2016).
51. Viswanathan, S. R. et al. Genome-scale analysis identifies paralog lethality as a vulnerability of chromosome 1p loss in cancer. Nat. Genet. 50, 937-943 (2018).
52. Kryukov, G. V. et al. MTAP deletion confers enhanced dependency on the PRMT5 arginine methyltransferase in cancer cells. Science 351, 1214-1218 (2016).
53. Neggers, J. E. et al. Synthetic lethal interaction between the ESCRT paralog enzymes VPS4A and VPS4B in cancers harboring loss of chromosome 18q or 16q. Cell Rep. 33, 108493 (2020).
54. Nilsson, R. et al. Metabolic enzyme expression highlights a key role for MTHFD2 and the mitochondrial folate pathway in cancer. Nat. Commun. 5, 3128 (2014).
55. Wei, Y. et al. Te efect of MTHFD2 on the proliferation and migration of colorectal cancer cell lines. Onco Targets Ter. 12, 6361-6370 (2019).
56. Wan, X. et al. Cisplatin inhibits SIRT3-deacetylation MTHFD2 to disturb cellular redox balance in colorectal cancer cell. Cell Death Dis. 11, 649 (2020).
57. Shukla, K. et al. MTHFD2 blockade enhances the efcacy of β-lapachone chemotherapy with ionizing radiation in head and neck squamous cell cancer. Front. Oncol. 10, 536377 (2020).
58. Ben-Sahra, I., Hoxhaj, G., Ricoult, S. J. H., Asara, J. M. & Manning, B. D. mTORCI induces purine synthesis through control of the mitochondrial tetrahydrofolate cycle. Science 351, 728-733 (2016).
59. Bindra, S. et al. Mitochondria in epithelial ovarian carcinoma exhibit abnormal phenotypes and blunted associations with biobehavioral factors. Sci. Rep. 11, 11595 (2021).
60. Judge, M. T. et al. Continuous in vivo metabolism by NMR. Front. Mol. Biosci. https://doi.org/10.3389/fmolb.2019.00026 (2019).
61. Cerami, E. et al. Te cBio cancer genomics portal: an open platform for exploring multidimensional cancer genomics data. Cancer Discov. 2, 401-404 (2012).
62. Gao, J. et al. Integrative analysis of complex cancer genomics and clinical profles using the cBioPortal. Sci. Signal. 6, pl1 (2013).
63. AACR Project GENIE Consortium. AACR Project GENIE: powering precision medicine through an international consortium. Cancer Discov. 7, 818-831 (2017).
64. Reich, M. et al. GenePattern 2.0. Nat. Genet. 38, 500-501 (2006).
65. Krzywinski, M. I. et al. Circos: an information aesthetic for comparative genomics. Genome Res. 19, 1639-1645 (2009).
66. Creed, J. H., Gerke, T. A. & Berglund, A. E. MatSurv: survival analysis and visualization in MATLAB. J. Open Source Sofw. https://doi.org/10.21105/joss.01830 (2020).
67. Moftt, R. A. et al. Virtual microdissection identifes distinct tumor- and stroma-specifc subtypes of pancreatic ductal adenocarcinoma. Nat. Genet. 47, 1168-1178 (2015).
68. Tate, J. G. et al. COSMIC: the catalogue of somatic mutations in cancer. Nucleic Acids Res. 47, D941-D947 (2018).
69. Conway, J. R., Lex, A. & Gehlenborg, N. UpSetR: an R package for the visualization of intersecting sets and their properties. Bioinformatics 33, 2938-2940 (2017).
70. Nagrath, D. et al. Sof constraints-based multiobjective framework for fux balance analysis. Metab. Eng. 12, 429-445 (2010).
71. Nagrath, D. et al. Integrated energy and fux balance based multiobjective framework for large-scale metabolic networks. Ann. Biomed. Eng. 35, 863-885 (2007).
72. Heirendt, L. et al. Creation and analysis of biochemical constraint-based models using the COBRA Toolbox v.3.0. Nat. Protoc. 14, 639-702 (2019).
73. Messac, A., Ismail-Yahaya, A. & Mattson, C. A. Te normalized normal constraint method for generating the Pareto frontier. Struct. Multidiscip. Optim. 25, 86-98 (2003).
74. Chen, T. & Guestrin, C. XGBoost: A scalable tree boosting system. In Proc. 22nd ACM SIGKDD International Conference on Knowledge Discovery and Data Mining (eds Krishnapuram, B. et al.) 785-794 (ACM Digital, 2016).
75. Achreja, A., Meurs, N. & Nagrath, D. Quantifying metabolic transfer mediated by extracellular vesicles using Exo-MFA: an integrated empirical and computational platform. Methods Mol. Biol. 2088, 205-221 (2020).
76. Achreja, A. et al. Exo-MFA—a 13C metabolic fux analysis framework to dissect tumor microenvironment-secreted exosome contributions towards cancer cell metabolism. Metab. Eng. 43, 156-172 (2017).
77. Yang, L. et al. Metabolic shifs toward glutamine regulate tumor growth, invasion and bioenergetics in ovarian cancer. Mol. Syst. Biol. 10, 728 (2014).
78. Zhao, H. et al. Tumor microenvironment derived exosomes pleiotropically modulate cancer cell metabolism. eLife 5, e10250 (2016).
79. Zhu, Z. et al. Tumour-reprogrammed stromal BCAT1 fuels branched-chain ketoacid dependency in stromal-rich PDAC tumours. Nat. Metab. https://doi.org/10.1038/s42255-020-0226-5 (2020).
80. Izar, B. et al. A single-cell landscape of high-grade serous ovarian cancer. Nat. Med. 26, 1271-1279 (2020).
81. Van Dijk, D. et al. Recovering gene interactions from single-cell data using data difusion. Cell 174, 716-729 (2018).
82. Moon, K. R. et al. Visualizing structure and transitions in high-dimensional biological data. Nat. Biotechnol. 37, 1482-1492 (2019).
83. Wickham, H. et al. ggplot2: Elegant Graphics for Data Analysis 2nd edn. Use R! (Springer-Verlag, 2016).
84. Cerami, E. et al. Te cBio cancer genomics portal: an open platform for exploring multidimensional cancer genomics data. Cancer Discov. 2, 401-404 (2012); correction 2, 960 (2012).
85. Shih, A. J. et al. Identification of grade and origin specific cell populations in serous epithelial ovarian cancer by single cell RNA-seq. PLoS ONE 13, e0206785 (2018).
86. Satija, R., Farrell, J. A., Gennert, D., Schier, A. F. & Regev, A. Spatial reconstruction of single-cell gene expression data. Nat. Biotechnol. 33, 495-502 (2015).
87. Newman, A. M. et al. Determining cell type abundance and expression from bulk tissues with digital cytometry. Nat. Biotechnol. 37, 773-782 (2019).
88. Yeung, T. L. et al. Systematic identification of druggable epithelial-stromal crosstalk signaling networks in ovarian cancer. J. Natl Cancer Inst. 111, 272-282 (2019).
89. Quek, L. E. & Turner, N. Using the human genome-scale metabolic model Recon 2 for steady-state fux analysis of cancer cell metabolism. Methods Mol. Biol. 1928, 479-489 (2019).

We/I claim:

1. A method for identifying collateral lethal genes or gene products, comprising: a) analyzing data derived from a cell having a first gene or gene product with a computer program to identify one or more collateral lethal genes or gene products from a paralogous metabolic pathway that provides metabolic flux compensation to said cell when said first gene has reduced or eliminated expression; and b) displaying processed data revealing the identify of said one or more collateral lethal genes or gene products.

2. The method of claim 1, wherein said lethal gene, when expressed or overexpressed, contributes to cancer.

3. The method of claim 1, wherein reduced or eliminated expression of said first gene results in ETC impairment in said cell.

4. The method for treating a subject comprising:
a) identifying a subject having ETC impairment; and
b) administering an MTHFD2 inhibitor to said subject.

5. The method of claim 4, wherein said ETC impairment comprises Complex 1 dysfunction.

6. The method of claim 4, wherein said ETC impairment is caused by reduced UQCR11 expression.

7. The method of claim 6, wherein said subject has UQCR11 copy loss.

8. The method of claim 6, wherein said UQCR11 expression is reduced in an ovarian cell of said subject.

9. The method of claim 8, wherein said ovarian cell is an ovarian cancer cell.

10. A method comprising: measuring MTHFD2 expression in a cell having ETC impairment.

11. The method of claim 10, wherein the ETC impairment is caused by reduced or eliminated expression of a gene that is not MTHFD2.

12. The method of claim 11, wherein said gene is UQCR11.

13. The method of claim 10, wherein said cell is an ovarian cell.

14. A system comprising a computer processor configured to a) analyze data derived from a cell having a first gene or gene product to identify one or more collateral lethal genes or gene products from a paralogous metabolic pathway that provides metabolic flux compensation to said cell when said first gene has reduced or eliminated expression; and/or b) display processed data revealing the identify of said one or more collateral lethal genes or gene products.
